(19) United States
(12) Patent Application Publication
(10) Pub. No.: US 2022/0383236 A1
(43) Pub. Date: Dec. 1, 2022

(54) Enterprise Workload Sharing System

(71) Applicant: CVS Pharmacy, Inc., Woonsocket, RI (US)

(72) Inventors: Steven W. Beede, Chester, NJ (US); Ajay K. Behuria, Upton, MA (US); Summer L. Widrick, Cranston, RI (US); Graham S. Cabrey, East Brookfield, MA (US); Paul R. Genuario, Cranston, RI (US); Sean Haggerty, Dedham, MA (US); Daniel G. LaCross, South Easton, MA (US); Kevin Pellerin, Douglas, MA (US); Ravi Vangala, Mead, NJ (US)

(21) Appl. No.: 17/885,069

(22) Filed: Aug. 10, 2022

Related U.S. Application Data
(62) Division of application No. 16/176,988, filed on Oct. 31, 2018.

(51) Int. Cl. G06Q 10/06; G16H 20/10

(52) U.S. Cl. CPC G06Q 10/063118; G06Q 10/0633; G16H 20/10

(57) ABSTRACT

Systems and methods are disclosed for enterprise work sharing between source and processing locations. For example, an enterprise work share engine assigns shared work requests to a processing pharmacy for prescription verification tasks that are prerequisites for prescription fulfillment at a source pharmacy. The assigned prescription verification task is completed by a pharmacy resource at the processing pharmacy and the disposition is sent to the source pharmacy. The prescription fulfillment task is completed by a pharmacy resource at the source pharmacy in response to receiving the disposition of the prescription verification task.

### CROSS REFERENCE TO RELATED APPLICATIONS

The present application is a divisional of U.S. patent application Ser. No. 16/176,988, filed Oct. 31, 2018, titled “Enterprise Workload Sharing System,” the entirety of which is hereby incorporated by reference.

### BACKGROUND

The present disclosure relates to workload sharing across multiple locations mediated by enterprise data systems. In particular, the present disclosure relates to sharing expert data review and validation (e.g., prescription verification by pharmacists) across independently managed locations (e.g., retail pharmacies serving different geographic locations) for enterprise-level load balancing, resource utilization, and performance metrics.

Conventionally, when a patient or doctor submits a prescription to be filled at a pharmacy, the prescription is reviewed and validated by a pharmacist to confirm appropriateness prior to filling the prescription. For example, appropriateness review may include determining whether the selected drug is generally prescribed for the patient's medical condition, age, and other factors, whether the dose, frequency, and route of administration align with the appropriate use and each other, whether there could be therapeutic duplication, and whether there are possible drug interactions. Errors, ambiguities, relevant questions for the prescribing physician or patient, and/or additional clarification, instructions, or safeguards for filling the prescription or providing instructions to the patient may be resolved or initiated through this verification step.

In some pharmacy work flows and related information systems, the prescription verification step may be handled at the same time as filling the prescription by the same pharmacists. In other pharmacies, the prescription verification step may be disaggregated from the prescription filling process (actual dispensing of medications, preparing as necessary, and packaging and documenting for patient pickup/delivery). This may give a single pharmacist greater flexibility in managing his or her work queue for efficiency, as well as allow multiple pharmacy resources to assist with separate work tasks. Similarly, patient and prescription data entry, prescription pickup, and other tasks may be shared with non-pharmacist staff, such as pharmacy technicians. Pharmacies may implement information systems to assist in supporting these tasks and workflows for their pharmacist and non-pharmacist staff.

Retail pharmacies with multiple locations may implement varying levels of enterprise-wide data systems to assist in collecting metrics and managing the performance of retail locations. These separate locations may include some level of local management and control, as well as performance accounting and incentives, that are supported by enterprise-wide systems and processes. Standardization of processes and aggregation of retail performance data may support analytics, performance improvement, and management decision-making around staffing, operations, supply chain, promotions, and other considerations.

### SUMMARY

The techniques introduced herein overcome the deficiencies and limitations of the prior art at least in part by providing systems and methods for enterprise work sharing between source and processing locations. For example, an enterprise work share engine qualifies and assigns shared work requests to a processing pharmacy for prescription verification tasks that are prerequisites for production tasks at a source pharmacy. The assigned prescription verification task is completed by a pharmacy resource at the processing pharmacy and the disposition is sent to the source pharmacy. The production task is completed by a pharmacy resource at the source pharmacy in response to receiving the disposition of the prescription verification task.

In one embodiment, the present disclosure describes a computer-implemented method for enterprise work sharing. A computing device receives a shared work request from a first location, processes a set of qualification rules in response to receiving the shared work request, identifies a second location using a set of assignment rules in response to a successful qualification of the shared work request, and sends an assigned work request to the second location based on the shared work request. The assigned work request includes a recall deadline and the computing device automatically terminates the assigned work request in response to reaching the recall deadline before completion of the assigned work request.

In some embodiments, the shared work request may include work parameters for a prescription verification task. A second location computing device at the second location may receive the assigned work request based on the shared work request including work parameters for the prescription verification task. The second location computing device may process the assigned work request in response to input received from a second pharmacy resource at the second location and send a work disposition in response to processing the assigned work request. A first location computing device at the first location may receive the work disposition and display production review parameters in response to the work disposition. A first pharmacy resource at the first location may use the production review parameters to complete a prescription corresponding to the prescription verification task. The first location computing device may receive the prescription, processing the prescription to generate the work parameters for the prescription verification task, and send the shared work request including the work parameters. The first location computing device may terminate the shared work request in response to input from the first location and send a manual pullback notification in response to terminating the shared work request. The second location computing device may identify a protected data resource at the first location computing device and receive the protected data resource from the first location computing device. The protected data resource may be used in processing the assigned work request.

In some embodiments, processing the set of qualification rules may further comprise identifying shared work request attributes selected from work attributes, source attributes, decision attributes, and configuration attributes, using work request attributes to index at least one decision table, and determining whether the shared work request qualifies for sharing based on the at least one decision table. The computing device may calculate a work delivery time for the shared work request. The work delivery time may include a first work completion offset to enable additional work tasks to be completed at the first location in response to disposition of the assigned work request at the second location. The computing device may calculate the recall deadline for the assigned work request. The recall deadline may include a second work completion offset to enable work tasks associated with the assigned work request to be completed at the first location in response to automatically terminating the assigned work request. The second location computing device may receive the assigned work request and the assigned work request may include the work delivery time, the recall deadline, and an estimated work time. The second location computing device may insert the assigned work request into a second location work queue based on the work delivery time, the recall deadline, the estimated work time, and pre-existing tasks in the second location work queue. The second location computing device may process the assigned work request in response to input received from a second pharmacy resource at the second location when the assigned work request is selected from the second location work queue and sending a work disposition in response to processing the assigned work request.

In some embodiments, the computing device may receive periodic capacity notifications from the second location, calculate a capacity banding model configured to project a second location workload across a plurality of future timebands based on the periodic capacity notifications, and determine whether the shared work request is assignable to the second location using the capacity banding model. The periodic capacity notifications may be received at a first time interval and the plurality of future timebands may each include a second time interval. The first time interval may be less than the second time interval. The second location computing device may generate a graphical user interface displaying a prescription verification task based on the assigned work request. The graphical user interface may be configured to display prescription verification data and a plurality of selectable disposition options for the prescription verification task. The computing device may log the shared work request from the first location and the assigned work request to the second location.

In one embodiment, the present disclosure describes a system for enterprise work sharing comprising at least one processor, at least one memory, and a pharmacy information systems interface configured to communicate with a source pharmacy and a processing pharmacy over a network. A shared work rules engine is stored in the at least one memory and configured for execution by the at least one processor to receive a shared work request from the source pharmacy, process a set of qualification rules in response to receiving the shared work request, identify the processing pharmacy using a set of assignment rules in response to a successful qualification of the shared work request, and send an assigned work request to the processing pharmacy based on the shared work request.

In some embodiments, a processing computing device at the processing pharmacy may be in communication with the shared work rules engine. The processing computing device may be configured for receiving the assigned work request based on the shared work request. The shared work request may include work parameters for a prescription verification task. The processing computing device may process the assigned work request in response to input received from a processing pharmacy resource at the processing pharmacy to complete the prescription verification task and send a work disposition in response to processing the assigned work request. The assigned work request may include a work delivery time, a recall deadline, and an estimated work time. The processing computing device may be further configured for inserting the assigned work request into a processing pharmacy work queue based on the work delivery time, the recall deadline, the estimated work time, and pre-existing tasks in the processing pharmacy work queue and selecting the assigned work request from the second location work queue for processing.

In some embodiments, a source computing device at the source pharmacy may be in communication with the shared work rules engine. The source computing device may be configured for receiving a prescription, processing the prescription to generate work parameters for a prescription verification task, sending the shared work request. The shared work request may include the work parameters. The source computing device may receive the work disposition and display production review parameters for the prescription in response to the work disposition. A source pharmacy resource at the source pharmacy may use the production review parameters to complete the prescription corresponding to the prescription verification task. The source computing device may be further configured for terminating the shared work request in response to input at the source pharmacy and sending a manual pullback notification in response to terminating the shared work request.

In some embodiments the shared work rules engine may be further configured for identifying shared work request attributes selected from work attributes, source attributes, decision attributes, and configuration attributes, using work request attributes to index at least one decision table, and determining whether the shared work request qualifies for sharing based on the at least one decision table. The shared work rules engine may be further configured for calculating a work delivery time for the shared work request and a recall deadline for the assigned work request. The work delivery time may include a first work completion offset to enable additional work tasks to be completed at the source pharmacy in response to disposition of the assigned work request at the processing pharmacy. The shared work request may be automatically terminated in response to reaching the recall deadline before completion of the assigned work request. The recall deadline may include a second work completion offset to enable work tasks associated with the assigned work request to be completed at the source pharmacy in response to automatically terminating the assigned work request.

In some embodiments, the shared work rules engine may be further configured for receiving periodic capacity notifications from the processing pharmacy, calculating a capacity banding model configured to project a second location workload across a plurality of future timebands based on the periodic capacity notifications, and determining whether the shared work request is assignable to the second location using the capacity banding model. The periodic capacity notifications may be received at a first time interval and the plurality of future timebands each include a second time interval. The first time interval may be less than the second time interval. The system may further comprise a shared work log. The shared work rules engine may be further configured for logging the shared work request from the source pharmacy to the shared work log and logging the assigned work request sent to the processing pharmacy to the shared work log.

In some embodiments, the system may further comprise a source computing device at the source pharmacy in communication with the shared work rules engine and a processing computing device at the processing pharmacy in communication with the shared work rules engine. The source computing device may be configured for receiving a prescription and storing a protected data resource related to the prescription. The processing computing device may be configured for receiving the assigned work request based on the shared work request, identifying the protected data resource at the source computing device, and receiving the protected data resource from the source computing device. The shared work request may include work parameters for a pharmacy task for the prescription. The protected data resource may not pass through the shared work rules engine.

In one embodiment, the present disclosure describes a system for enterprise work sharing comprising an enterprise computing device, a processing pharmacy computing device, and a source pharmacy computing device. The enterprise computing device is configured to generate and assign a shared work request for a prescription verification task. The prescription verification task is a prerequisite task for a production task at a source pharmacy. The processing pharmacy computing device at a processing pharmacy is configured to generate a processing graphical user interface displaying the prescription verification task assigned by the enterprise computing system. The processing graphical user interface is configured to display prescription verification data and a plurality of selectable disposition options for the prescription verification task. The source pharmacy computing device at the source pharmacy is configured to generate a production graphical user interface displaying the production task in response to receiving a disposition of the prescription verification task.

The various embodiments advantageously apply the teachings of prescription processing systems to improve the functionality of such computer systems. The various embodiments include operations to overcome or at least reduce the issues in the previous prescription processing systems discussed above and, accordingly, are more efficient and reliable for sharing workloads across locations, while enabling remote processing of expert tasks, such as pharmacist prescription verification review. That is, the various embodiments disclosed herein include hardware and/or software with functionality to improve the processing of pharmacy tasks at pharmacies other than the pharmacy filling the prescription, based on managing capacity and assignments among pharmacy locations. Accordingly, the embodiments disclosed herein provide various improvements to prescription processing systems.

It should be understood that language used in the present disclosure has been principally selected for readability and instructional purposes, and not to limit the scope of the subject matter disclosed herein.

### BRIEF DESCRIPTION OF THE DRAWINGS

The present disclosure is illustrated by way of example, and not by way of limitation in the figures of the accompanying drawings in which like reference numerals are used to refer to similar elements.

FIG. 1 is a block diagram of an example enterprise workload sharing system in accordance with an embodiment.

FIG. 2 is a block diagram of an example source pharmacy computing device in accordance with an embodiment.

FIG. 3 is a block diagram of an example processing pharmacy computing device in accordance with an embodiment.

FIG. 4 is a block diagram of a portion of an example enterprise workload sharing system, such as the system in FIG. 1, in accordance with an embodiment.

FIG. 5 is a block diagram of a portion of an example enterprise workload sharing system, such as the system in FIG. 1, in accordance with an embodiment.

FIG. 6 is a block diagram of a portion of an example enterprise workload sharing system, such as the system in FIG. 1, in accordance with an embodiment.

FIG. 7 is a flowchart of an example method for enterprise workload sharing in accordance with an embodiment.

FIG. 8 is a flowchart of an example method for a pharmacy sharing work in accordance with an embodiment.

FIG. 9 is a flowchart of an example method for qualifying and assigning shared work in accordance with an embodiment.

FIG. 10 is a flowchart of an example method for a pharmacy processing shared work in accordance with an embodiment.

FIG. 11 is a flowchart of an example method for pharmacy capacity notifications in accordance with an embodiment.

FIG. 12 illustrates an example of a shared work dashboard and local work queue in accordance with an embodiment.

FIG. 13 illustrates an example of a shared work processing screen in accordance with an embodiment.

### DETAILED DESCRIPTION

With reference to the figures, reference numbers may be used to refer to components found in any of the figures, regardless whether those reference numbers are shown in the figure being described. Further, where a reference number includes a decimal referring to one of multiple similar components (e.g., component 000.1, 000.2, and 000.3), the reference number may be used without the decimal to refer to one or all of the similar components.

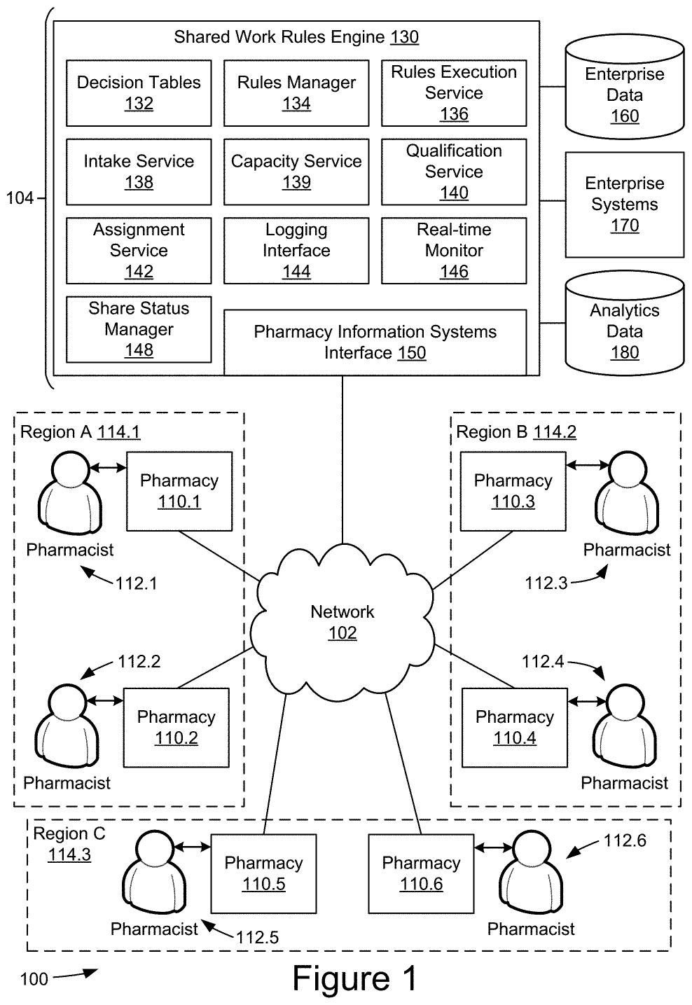

FIG. 1 is a block diagram of an example system 100 for enterprise workload sharing in accordance with an embodiment. The illustrated system 100 may include an enterprise service layer 104 associated with supporting information systems for a plurality of retail locations, which in one embodiment are retail pharmacies. In some embodiments, system 100 may include any number of pharmacies 110 respectively staffed by pharmacists 112 and in communication with enterprise service layer 104 over network 102. In some embodiments, enterprise service layer 104 may include a shared work rules engine 130 for managing workload sharing between two or more pharmacies 110. Enterprise service layer 104 may include any number of additional modules or subsystems to support workload sharing and other pharmacy functions, such as enterprise data store 160 and enterprise systems 170. Network 102 provides for electronic communication to support interaction of the different components with one another, although other system configurations are possible including other devices, systems, and networks.

In some embodiments, a source pharmacy, such as pharmacy 110.1, may identify prescriptions that may be verified for appropriateness by a pharmacist at another location, such as pharmacy 110.3. The source pharmacy may enable workload sharing or and the source pharmacy computing device may select prescriptions to be shared. Prescriptions meeting certain pre-qualification criteria may be sent to shared work rules engine 130 for qualification and assignment. If the prescription qualifies and can be assigned to an eligible processing pharmacy, the prescription is provided to a processing pharmacy, such as pharmacy 110.3. The prescription verification task may be added to the local work queue at the processing pharmacy and processed by a pharmacist there when it is selected from the queue. The disposition may then be provided back to the source pharmacy and the pharmacy resource there may complete the production fill and preparation of patient instructions (or other tasks determined by the disposition) in accordance with the remaining workflow. Shared work rules engine 130 may provide enterprise-level oversight of this process to assure that only appropriate work tasks are shared, workloads are spread throughout the day for source pharmacies, and spread across processing pharmacies, remote processing is handled in a timely manner, metrics are collected and tracked regarding quotas and performance, and source pharmacies are assured of the necessary control to support customer inquiries and changes.

While the shared work examples provided throughout this description relate to prescription verification for appropriateness by a remote pharmacist, other pharmacy work tasks may be configured for shared work using similar systems, methods, and appropriate pharmacy resources (e.g., pharmacists, pharmacy technicians, etc.). For example, data entry and data entry verification tasks may be shared among technicians or other staff. Visual verifications of prescription medications and associated documentation may be verified through use of images taken at the source location and shared with a processing location.

Network 102 may include any number of networks and/or network types. For example, the network 102 may include, but is not limited to, one or more local area networks (LANs), wide area networks (WANs) (e.g., the Internet), virtual private networks (VPNs), wireless wide area network (WWANs), WiMAX® networks, personal area networks (PANs) (e.g., Bluetooth® communication networks), various combinations thereof, etc. These private and/or public networks may have any number of configurations and/or topologies, and data may be transmitted via the networks using a variety of different communication protocols including, for example, various Internet layer, transport layer, or application layer protocols. For example, data may be transmitted via the networks using TCP/IP, UDP, TCP, HTTP, HTTPS, DASH, RTSP, RTP, RTCP, VOIP, FTP, WS, WAP, SMS, MMS, XMS, IMAP, SMTP, POP, WebDAV, or other known protocols.

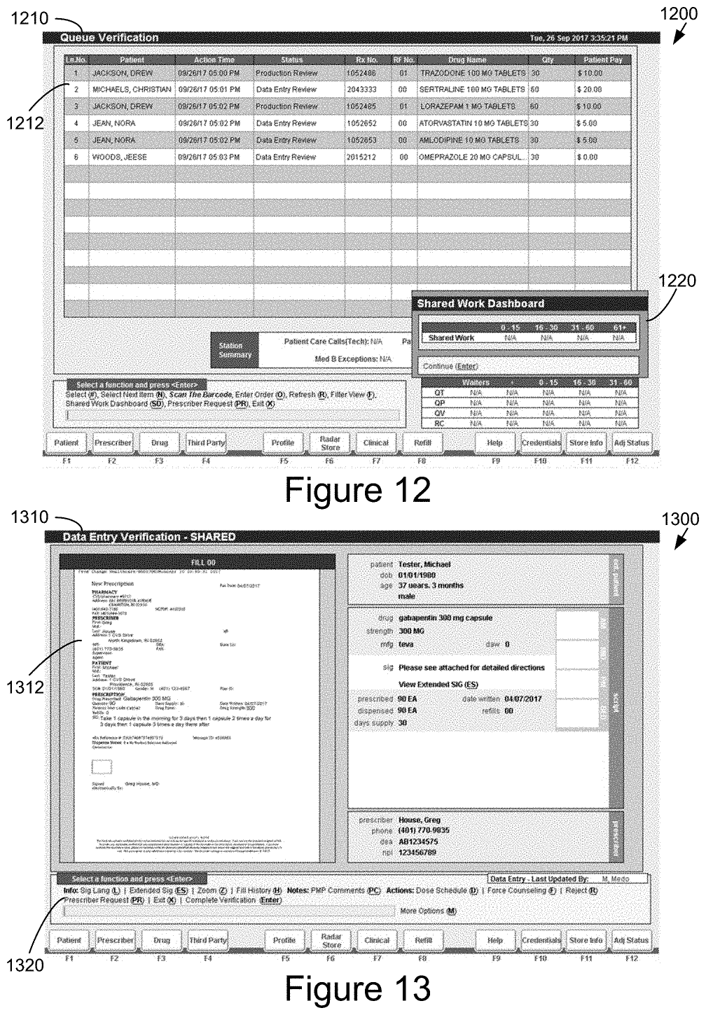

Each pharmacy 110 may include one or more computing devices through which pharmacists 112 and other pharmacy staff interact with enterprise service layer 104. For example, the computing devices at pharmacies 110 may run or access a pharmacy web portal that includes one or more functions supported by enterprise service layer 104. In some embodiments, the pharmacy web portal provides or supports a variety of functions within pharmacies 110 and may be accessed by pharmacists 112 and other staff to support their work functions. In some embodiments, the pharmacy web portal includes data and workflow management for intaking, processing, and filling prescriptions. For example, the pharmacy web portal may provide one or more interfaces for receiving or entering patient information and prescription information, verification of data entry and prescription appropriateness verification, preparing and filling prescriptions and related patient instructions, and order pickup or delivery. In some embodiments, the pharmacy web portal may include workload sharing functions and interfaces for source pharmacies and processing pharmacies. Example interfaces for a pharmacy web portal that supports workload sharing is further described below with regard to FIGS. 12 and 13.

In some embodiments, pharmacies 110 may be spread across a plurality of regions 114. For example, pharmacy 110.1 and pharmacy 110.2 may be in region A 114.1, pharmacy 110.3 and pharmacy 110.4 may be in region B 114.2, and pharmacy 110.5 and pharmacy 110.6 may be in region C 114.3. Each pharmacy 110 may be in geographically separate physical locations, such as different retail pharmacy locations with different street addresses. In some embodiments, regions may designate geographic areas with different constraints on regulated operations. For example, pharmacies and pharmacists may be accredited and regulated by national, state, or other regional jurisdictions. In the context of workload sharing, specific regulations on patient data, controlled substances, insurance requirements, licensing and other qualifications for specific tasks, and the types of data and tasks that can be shared across regional lines may impacted by regulatory differences among regions. For example, certain states or other jurisdictions may limit the ability to transfer tasks to other states due to state-specific licensing requirements. Further, individual licensing and credentials of pharmacists and/or other staff in each pharmacy may impact sharing between locations and/or regions. In some embodiments, rules for sharing based on source and processing regions and/or staff credentials may be included in shared work rules engine 130.

In some embodiments, shared work rules engine 130 may operate within enterprise service layer 104 to support shared work functions at participating pharmacies. For example, shared work rules engine 130 may be built on top of existing enterprise data system architecture, such as enterprise data store 160, enterprise systems 170, and analytics data store 180. Enterprise data store 160 may include one or more enterprise databases or storage networks aggregating data from various enterprise data sources, including retail locations, supply chain management, operations, and strategic management.

Example enterprise systems 170 may include retail analytics systems, workforce management systems, pharmacy workflow systems, and other systems for supporting one or more corporate operations across retail locations. For example, retail analytics systems may include one or more data analytics, management, and/or planning tools that track and analyze retail sales, customer, and supply chain data to inform purchasing, promotion, management, and other decisions, often utilizing enterprise data store 160. Workforce management systems may include one or more data analytics, management, and/or planning tools that track and analyze human resource and performance data to inform staffing, hiring, compensation, capacity planning, and other decisions, often utilizing enterprise data store 160. Pharmacy workflow systems may provide standardized data resources and processes for supporting common work tasks in pharmacies 110, including receiving or entering patient information and prescription information, verification of data entry and prescription appropriateness verification, preparing and filling prescriptions and related patient instructions, and order pickup or delivery. In some embodiments, shared work rules engine 130 may integrate with or extend the capabilities of an existing system, such as pharmacy workflow systems.

In some embodiments, analytics data store 180 may include analytical data sets extracted or processed from enterprise data store 160 and/or other data sources. For example, analytics data store 180 may include query, modeling, baseline, and similar data sets related to business intelligence and strategic management analyses. In some embodiments, shared work rules engine 130 may be supported by analytics data store 180 for rules generation and maintenance.

In some embodiments, shared work rules engine 130 may include various data and/or service modules for completing one or more tasks to support enterprise workload sharing. For example, share work rules engine 130 may include decision tables 132, rules manager 134, rules execution service 136, intake service 138, capacity service 139, qualification service 140, assignment service 142, logging interface 144, real-time monitor 146, and share status manager 148. In some embodiments, shared work rules engine 130 may include a pharmacy information systems interface 150 for communicating with computing devices at pharmacies 110. For example, enterprise service layer 104 may include one or more messaging platforms for communication between enterprise service layer 104 and computing systems in pharmacies 110. In some embodiments, shared work rules engine 130 may utilize the same pharmacy information systems interface 150 as pharmacy workflow systems.

In some embodiments, pharmacy information systems interface 150 may be embodied in a web server that is accessed by computing systems at pharmacies 110. A web server may include computer logic executable by the enterprise service layer 104 to manage content requests. The web server may include an HTTP server, a REST (representational state transfer) service, or another suitable server type. The web server may receive content requests (e.g., object search requests, HTTP requests) from computing devices to provide the pharmacy web portal and/or provide content for the pharmacy web portal, although other configurations are also possible and contemplated. In some instances, the web server may format the content using a web language and provide the pharmacy web portal content to a corresponding application or web browser (not shown) for processing and/or rendering to the user for display. The web server may be coupled to enterprise data store 160 to store retrieve, and/or manipulate data stored therein and may be coupled to the pharmacies 110 to facilitate their operations.

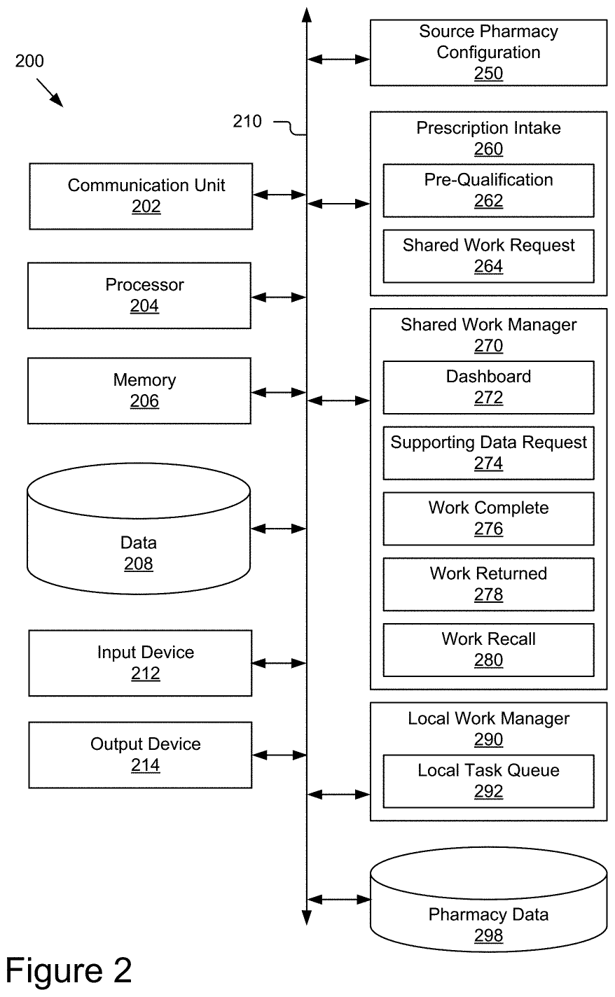

As depicted in the example of FIG. 2, a computing system 200 may include and/or access one or more data sources and/or functions to facilitate workload sharing at a source pharmacy. For example, computing system 200 may provide a pharmacy workflow application and/or interact with a pharmacy web portal to enable users at the source pharmacy to receive or enter patient information and prescription information, verify data entry, verify prescription appropriateness, prepare and fill prescriptions and related patient instructions, and manage order pickup or delivery. In some embodiments, computing system 200 allows the source pharmacy to share one or more tasks from their workflow for completion at another location.

As depicted, the computing system 200 may include a communication unit 202, a processor 204, a memory 206, a data store 208, an input device 212, and an output device 214, which may be communicatively coupled by a communication bus 210. Computing system 200 depicted in FIG. 2 is provided by way of example and it should be understood that it may take other forms and include additional or fewer components without departing from the scope of the present disclosure. For instance, various components of the computing device may be coupled for communication using a variety of communication protocols and/or technologies including, for instance, communication buses, software communication mechanisms, computer networks, etc. While not shown, computing system 200 may include various operating systems, sensors, additional processors, and other physical configurations. The communication unit 202, processor 204, memory 206, etc., are representative of one or more of these components.

Processor 204 may execute software instructions by performing various input, logical, and/or mathematical operations. Processor 204 may have various computing architectures to method data signals (e.g., CISC, RISC, etc.). Processor 204 may be physical and/or virtual and may include a single core or plurality of processing units and/or cores. In some implementations, processor 204 may be coupled to memory 206 via bus 210 to access data and instructions therefrom and store data therein. Bus 210 may couple processor 204 to the other components of the computing system 200 including, for example, memory 206, communication unit 202, input device 212, output device 214, and data store 208.

Memory 206 may store and provide access to data to the other components of computing system 200. Memory 206 may be included in a single computing device or a plurality of computing devices. In some implementations, memory 206 may store instructions and/or data that may be executed by processor 204. For example, the memory 206 may store one or more of a pharmacy workflow application, pharmacy web portal, and their respective components, depending on the configuration. Memory 206 is also capable of storing other instructions and data, including, for example, an operating system, hardware drivers, other software applications, databases, etc. Memory 206 may be coupled to the bus 210 for communication with the processor 204 and the other components of computing system 200.

Memory 206 may include a non-transitory computer-usable (e.g., readable, writeable, etc.) medium, which can be any non-transitory apparatus or device that can contain, store, communicate, propagate or transport instructions, data, computer programs, software, code, routines, etc., for processing by or in connection with processor 204. Memory 206 may include computer program instructions for implementing methods of expedited admissions and medication delivery as described above. In some implementations, memory 206 may include one or more of volatile memory and non-volatile memory (e.g., RAM, ROM, hard disk, optical disk, etc.). It should be understood that the memory 206 may be a single device or may include multiple types of devices and configurations.

Bus 210 can include a communication bus for transferring data between components of a computing device or between computing devices, a network bus system including the network 102 or portions thereof, a processor mesh, a combination thereof, etc. In some implementations, prescription intake module 260, shared work manager 270, local work manager 290, and various other components operating on computing system 200 (operating systems, device drivers, etc.) may cooperate and communicate via a communication mechanism included in or implemented in association with bus 210. The software communication mechanism can include and/or facilitate, for example, inter-method communication, local function or procedure calls, remote procedure calls, an object broker (e.g., CORBA), direct socket communication (e.g., TCP/IP sockets) among software modules, UDP broadcasts and receipts, HTTP connections, etc. Further, any or all of the communication could be secure (e.g., SSH, HTTPS, etc.).

Communication unit 202 may include one or more interface devices (UF) for wired and wireless connectivity among the components of the system 100. For instance, communication unit 202 may include, but is not limited to, various types known connectivity and interface options. Communication unit 202 may be coupled to the other components of computing system 200 via bus 210. Communication unit 202 can provide other connections to network 102 and to other entities of system 100 using various standard communication protocols.

Input device 212 may include any device for inputting information into computing system 200. In some implementations, input device 212 may include one or more peripheral devices. For example, input device 212 may include a keyboard, a pointing device, microphone, an image/video capture device (e.g., camera), a touch-screen display integrated with the output device 214, etc. Output device 214 may be any device capable of outputting information from the computing system 200. Output device 214 may include one or more of a display (LCD, OLED, etc.), a printer, a haptic device, an audio reproduction device, a touch-screen display, a remote computing device, etc. In some implementations, the output device is a display which may display electronic images and data output by a processor of the computing system 200 for presentation to a user, such as the processor 204 or another dedicated processor.

Data store 208 may include information sources for storing and providing access to data. In some implementations, data store 208 may store data associated with a database management system (DBMS) operable on the computing system 200. For example, the DBMS could include a structured query language (SQL) DBMS, a NoSQL DMBS, various combinations thereof, etc. In some instances, the DBMS may store data in multi-dimensional tables comprised of rows and columns, and manipulate, (e.g., insert, query, update and/or delete), rows of data using programmatic operations.

The data stored by data store 208 may be organized and queried using various criteria including any type of data stored by them, such as a user identifier, a prescription record identifier, user attributes, pharmacy item attributes, retail item attributes, store identification, location data, etc. Data store 208 may include data tables, databases, or other organized collections of data.

Data store 208 may be included in computing system 200 or in another computing system and/or storage system distinct from but coupled to or accessible by computing system 200. Data store 208 can include one or more non-transitory computer-readable mediums for storing the data. In some implementations, data store 208 may be incorporated with the memory 206 or may be distinct therefrom.

The components 202, 204, 206, 208, 212, and/or 214 may be communicatively coupled by bus 210 and/or processor 204 to one another and/or the other components of computing system 200. In some implementations, the components 202, 204, 206, 208, 212, and/or 214 may include computer logic (e.g., software logic, hardware logic, etc.) executable by the processor 204 to provide their acts and/or functionality. In any of the foregoing implementations, these components 202, 204, 206, 208, 212, and/or 214 may be adapted for cooperation and communication with processor 204 and the other components of the computing system 200.

Source pharmacy configuration 250 may include a plurality of source pharmacy configuration parameters for use by other portions of the enterprise workload sharing architecture. For example, source pharmacy configuration 250 may include a configuration file or other data structure for storing information descriptive of the store operations and/or its availability for workload sharing. In some embodiments, source pharmacy configuration 250 may include store operation parameters, such as store hours, pharmacy hours, related services, etc., staffing parameters, such as staff numbers, position types, shifts, etc., and/or configuration settings specific to workload sharing, such as work/task types, hours, capacity, qualification constraints/requirements, etc. In some embodiments, a pharmacy may be both a source pharmacy and processing pharmacy and source pharmacy configuration 250 may also include processing pharmacy parameters.

Prescription intake module 260 may include one or more workflows for receiving prescriptions to be filled and delivered at or from the source pharmacy. For example, a source pharmacy may have an existing pharmacy work flow application or pharmacy web portal used for managing the data entry, processing, data support, and various tasks related to filling a prescription. In some embodiments, prescription intake module 260 may include a user or system interface for receiving prescription information, such as directly from a patient with a written script, through a healthcare information system or healthcare data exchange format, from another pharmacy or enterprise prescription handling layer, etc. In some embodiments, prescription intake module 260 may also include intake and/or updating of patient/customer information and/or insurance information related to the prescription. For example, prescription, patient, customer, insurance, and healthcare provider records may be generated or updated and stored in pharmacy data store 298 and/or an enterprise data store, such as enterprise data store 160 in FIG. 1.

Prescription intake module 260 may include pre-qualification module 262 and shared work request module 264 for processing incoming prescriptions for possible workload sharing opportunities. In some embodiments, all incoming prescriptions may be processed through pre-qualification module 262 during periods when source work share status is enabled. In some embodiments, a graphical user interface provided through computing system 200 may enable a user, such as a pharmacy technician or pharmacist doing prescription entry, data review, or work queue management, to either select prescriptions as candidates for work share and/or select prescriptions to be excluded from work share. For example, a pharmacy web portal may provide one or more screens for reviewing prescriptions to be filled at the source pharmacy, such as by status, by work task, by patient, or assembled in a local task queue 292.

Pre-qualification module 262 may include logic and data for making an initial determination of whether a given prescription or pharmacy task is a candidate for work share. For example, pre-qualification module 262 may include a series of parameters, conditions, or filters that must be met by a selected prescription to be eligible for possible work share assignment. In some embodiments, pre-qualification module 262 may include a flag or similar status designation for whether the source store is presently enabling work share. Other filters may include time constraints (rush prescriptions or prescriptions for a physically present and waiting customer), patient constraints, prescription type constraints, pharmacists constraints, capacity/utilization thresholds, etc. For example, pre-qualification module 262 may evaluate work share start and stop times and/or a stop sharing indicator, evaluate local staffing capacity and utilization based on the existing staff and local work queue, calculate a minimum buffer or threshold to the promised delivery time, and/or evaluate characteristics of the prescription (e.g., waiter fill, completion fill, on hold, partial fill, out of stock, etc.) or patient (e.g., patient opt out). In some embodiments, a series of filter conditions may be passed for a prescription to trigger a shared work request.

Shared work request module 264 may generate a shared work request for a work task to be processed by another location, such as another pharmacy. For example, shared work request module 264 may generate a shared work request message or function call to a shared work coordinator in the enterprise service layer, such as shared work rules engine 130 in FIG. 1. In some embodiments, shared work request module 264 may identify and/or assign shared work request parameters to a shared work request, such as work type, prescription attributes, and other information for qualifying, assigning, and/or completing an assigned work task. Shared work request module 264 may also generate a transaction identifier for tracking the individual shared work request and a return by time, generally calculated from the promised delivery time and an estimated work completion buffer to allow the source pharmacy to complete the prescription fill following disposition of the shared work task. In some embodiments, shared work request module 264 may update shared work manager 270 by providing the transaction ID, return by time, and other shared work request parameters. For example, one or more of these parameters or derivatives thereof may be displayed on shared work dashboard 272.

In some embodiments, shared work request module 264 may also wait for a response from a shared work coordinator, such as shared work rules engine 130. For example, the shared work coordinator may return a qualification decision, regarding whether the shared work request meets enterprise criteria for sharing and/or whether a processing pharmacy can be assigned. Shared work request module 264 may process a non-qualification decision by removing the shared work request and putting the work task into the local task queue 292. Shared work request module 264 may also update the shared work manager 270. In some embodiments, shared work request module 264 may receive a stop indicator from the shared work coordinator as part of a qualification decision or as an independent message. A stop indicator may be communicated to pre-qualification module 262 to stop further pre-qualifications until the stop indicator is removed. For example, a stop indicator may be received when a certain quota threshold for the source store or overall sharing is passed or available processing capacity drops below a certain level and the stop indicator may be removed when a new metric period begins and the stop condition is otherwise abated.

In some embodiments, shared work request module 264 may include a shared work request timer and request expiration time for calculating the expiration of a shared work request. In some embodiments, the shared work request timer may be invoked when the shared work request is sent and expire at a shared work completion deadline or return-by time. In some embodiments, the expiration logic may include a plurality of deadlines or thresholds, such as receiving the qualification decision, receiving an assignment decision, and receiving a disposition notification. In each case, failure to receive the notice prior to reaching the relevant expiration time may trigger termination of the shared work request, update to shared work manager 270, and reassignment of the work task to local task queue 292.

Shared work manager 270 may provide pharmacists and other personnel at the source pharmacy with information and functions related to one or more shared work requests that have been sent for processing. For example, shared work manager 270 may integrate with an existing pharmacy workflow application or pharmacy web portal to monitor and support shared work requests being completed at one or more processing pharmacies. In some embodiments, shared work manager 270 may include a shared work dashboard 272, supporting data request module 274, work complete module 276, work returned module 278, and work recall module 280.

In some embodiments, shared work dashboard 272 may include a graphical user interface displayed through output device 214 to provide one or more views of shared work request records or data. For example, shared work dashboard 272 may include a list of shared work requests issued by shared work request module 264. Shared work dashboard 272 may display one or more parameters of each shared work request, such as transaction ID, patient, prescription ID, prescription type, medication, customer delivery deadline, shared work completion deadline, shared work status, etc. In some embodiments, shared work dashboard 272 may provide controls for searching, filtering, and otherwise navigating shared work requests. For example, an initial screen may list only active shared work requests, but options to navigate historical shared work data (completed, returned, and recalled) may be available. In some embodiments, one or more summary views may aggregate and report the number of active shared work requests projected over time intervals based on their shared work completion deadlines or timebands as they relate to staffing capacity. In some embodiments, shared work dashboard 272 may be integrated with one or more other dashboards, such as work flow dashboard in local work manager 290, to enable a user to see shared work requests in the context of the local workload, such as local task queue 292.

In some embodiments, shared work requests may involve one or more data sources, some of which are sensitive and protected data resources, such as image files, patient records, prescription records, or other files or data structures, that may not be included in the parameters of the shared work request and/or may not be directly accessible to a processing pharmacy locally or through the enterprise service layer. For example, prescription image files may be stored at the source pharmacy and be identified as a protected data resource due to patient information. Shared work manager 270 may include supporting data request module 274 to enable data stored in or retrievable by the source pharmacy to be provided directly to the processing pharmacy on request. In some embodiments, shared data request module 274 may access one or more data sources, such as pharmacy data store 298 to provide the requested data. For example, a message may be received from the processing pharmacy requesting one or more data elements, such as image files, structured data, queries against a database, etc. Supporting data request module 274 may provide access to the requested data source and/or process the message and provide the requested data in a return message.

Work complete module 276 may receive a disposition notification that enables the source pharmacy to complete any remaining workflow related to the prescription or other shared task. For example, a shared work request for prescription verification may return a prescription verification complete message that is received and/or processed by work complete module 276. In some embodiments, work complete module 276 parses the specific determination made in the work complete message and determines the next task (or tasks) in the workflow that should be added to the local work queue. For example, work complete module 276 may receive a work complete notification that includes data verification approval, a prescriber request, a clinical clarification request, or a data verification rejection. Each of these dispositions may trigger a different follow-up work task in local task queue 292. For example, an approval may add a production review task, a prescriber request may add a call to the prescribing healthcare provider, a clinical clarification request may generate additional instructions for filling and/or delivering the prescription, and a rejection may trigger the pharmacy's process for rejecting prescription fill requests. Once the new task is generated, its position in local task queue 292 may be determined using the existing work flow logic and the scheduled delivery deadline as if the verification review had been conducted at the source pharmacy.

Work returned module 278 may respond to shared work requests that made it through prequalification and were sent for processing, but are returned to the source pharmacy without completion. For example, a shared work request may be returned or expire because a stop threshold is reached at the enterprise level (such as based on source pharmacy quotas or enterprise load balancing factors), no processing pharmacy is available for assignment, an assigned processing pharmacy elects to return the work, or one or more automatic return deadlines are reached. In some embodiments, work returned module 278 may respond to a return message received from the work share coordinator or processing pharmacy indicating that a shared work request is being returned without completion. In some embodiments, shared work request module 264 or another component of computing system 200 may generate a termination of a shared work request, such as in response to the expiration of one or more deadlines for various steps or confirmation messages. For example, work returned module 278 may be triggered by expiration of a shared work request timer. In response to receiving a work return notice or other termination of a shared work request, work returned module 278 may place the returned work item, such as a prescription verification task, in local task queue 292 for local completion.

Work recall module 280 may enable computing system 200 or a user thereof to trigger a conditional or manual recall of a shared work request. For example, the source pharmacy may receive a customer request to accelerate processing of a prescription or receive additional information regarding a patient or prescription that changes its qualifications for sharing. In some cases, the source pharmacy may have authority to recall shared work requests for any reason or when their one capacity exceeds the available work in queue. In some embodiments, shared work manager 270 may provide one or more interface elements for a user, such as a pharmacist, to select a work recall option, such as a link, button, menu option, toggle, or similar selector element. For example, the work recall option may be provided from shared work dashboard 272, local work manager 290, a customer relationship management tool, or a patient or prescription record. In some embodiments, work recall module 280 may include decision logic similar to prequalification module 262 for reevaluating changes in prescription information and automatically triggering recall of a shared work request. In response to a triggered recall, the shared work request may be terminated and the task places in local task queue 292. In some embodiments, a termination message may be sent to the processing pharmacy and/or shared work coordinator.

Local work manager 290 may include one or more systems for managing work tasks at the source pharmacy. For example, a source pharmacy may have an existing pharmacy work flow application or pharmacy web portal used for managing the data entry, processing, data support, and various tasks related to filling a prescription. Local task queue 292 may include one or more work flows and accumulated tasks for a pharmacist and/or other staff to complete in support of the local work flows, such as receiving, processing, and filling prescriptions. Local work manager 290 and local task queue 292 may include logic and parameters for prioritizing, navigating, and selecting work tasks. In some embodiments, follow-on tasks from complete shared work, returned work, and recalled work may processed and integrated into local task queue 292 as if the prior step had been completed at the source pharmacy.

Pharmacy data store 298 may include data collected by and/or related to the source pharmacy. For example, each pharmacy may collect and store retail and pharmacy operations data, such as point-of-sale data, supply chain data, pharmacy workflow data, employment data, etc. In some embodiments, pharmacy data store 298 may provide configuration and supporting data for source pharmacy configuration 250, prescription intake module 260, shared work manager 270, and local work manager 290. In some embodiments, pharmacy data store 298 may support additional systems that may interact with shared work functions, such as a retail analytics system and/or workforce management system.

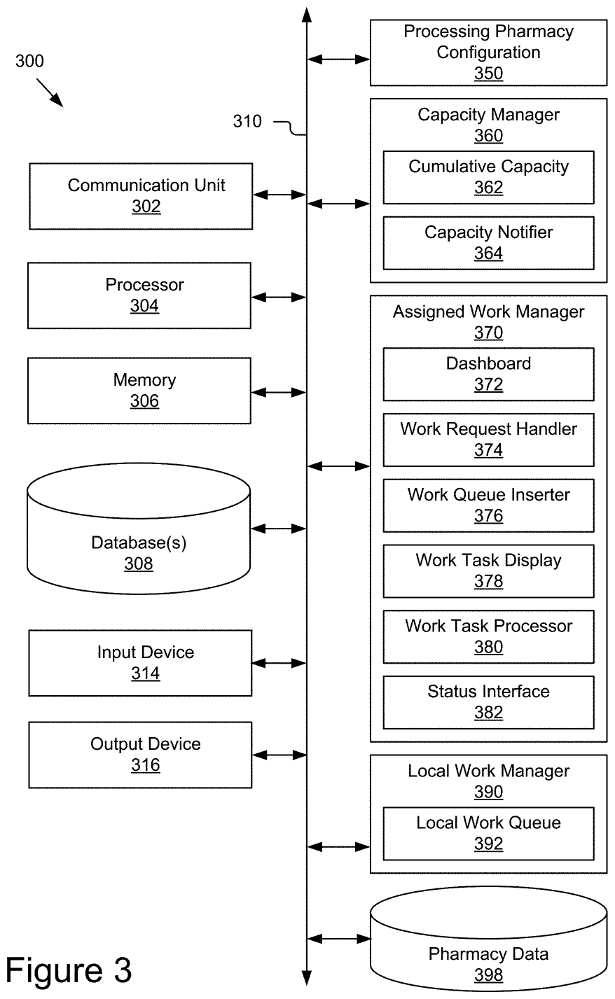

As depicted in the example of FIG. 3, a computing system 300 may include and/or access one or more data sources and/or functions to facilitate workload sharing at a processing pharmacy. For example, computing system 300 may provide a pharmacy workflow application and/or interact with a pharmacy web portal to enable users at the processing pharmacy to receive or enter patient information and prescription information, verify data entry, verify prescription appropriateness, prepare and fill prescriptions and related patient instructions, and manage order pickup or delivery for local prescriptions. In some embodiments, computing system 300 allows the processing pharmacy to utilize one or more tasks from their workflow for completion of a remote task, such as prescription verification, for a source pharmacy.

As depicted, computing system 300 may include a communication unit 302, a processor 304, a memory 306, a data store 308, an input device 312, and an output device 314, which may be communicatively coupled by a communication bus 310. Computing system 300 depicted in FIG. 3 is provided by way of example and it should be understood that it may take other forms and include additional or fewer components without departing from the scope of the present disclosure. For instance, various components of the computing device may be coupled for communication using a variety of communication protocols and/or technologies including, for instance, communication buses, software communication mechanisms, computer networks, etc. While not shown, computing system 300 may include various operating systems, sensors, additional processors, and other physical configurations. The communication unit 302, processor 304, memory 306, etc., are representative of one or more of these components. In some embodiments, computing system 300 and its components, such as communication unit 302, processor 304, memory 306, etc., may be selected, configured, and/or operate similarly to corresponding components described above with regard to computing system 200 in FIG. 2.

Processing pharmacy configuration 350 may include a plurality of processing pharmacy configuration parameters for use by other portions of the enterprise workload sharing architecture. For example, processing pharmacy configuration 350 may include a configuration file or other data structure for storing information descriptive of the store operations and/or its availability for workload sharing. In some embodiments, processing pharmacy configuration 350 may include store operation parameters, such as store hours, pharmacy hours, related services, etc., staffing parameters, such as staff numbers, position types, shifts, etc., and/or configuration settings specific to workload sharing, such as work/task types, hours, capacity, qualification constraints/requirements, task buffer time, etc. In some embodiments, processing pharmacy configuration 350 may include parameters related to capacity management, such as worker (e.g., pharmacy resource) utilization thresholds, capacity notification cadence or cycle time, etc. In some embodiments, a pharmacy may be both a source pharmacy and processing pharmacy and processing pharmacy configuration 350 may also include source pharmacy parameters.

Capacity manager 360 may provide capacity notifications to other components of the enterprise workload sharing architecture, such as a work share rules engine, capacity service, or one or more source pharmacies. For example, capacity manager 360 may monitor and publish capacity for one or more categories of tasks in terms of hours, worker-hours, task units, or some other unit of measure based on the staffing and availability within the processing pharmacy. In some embodiments, capacity manager 360 may provide dynamic, real-time information regarding the present availability based on actual (as oppose to scheduled) work capacity and local capacity allocations, such as an existing local work queue, reassignment of capacity to other tasks, etc. In some embodiments, capacity manager 360 may include a cumulative capacity module 362 and a capacity notifier 364.

Cumulative capacity module 362 may use the current staffing available and work commitments at a processing store to calculate capacity. For example, cumulative capacity module 362 may identify the staffing model for the processing store (e.g., worker number, worker type (e.g., pharmacist), and shift information), calculate the total capacity for the staff over a given period, and calculate the time required to complete the work tasks currently in the processing store's local work queue 392. In some embodiments, cumulative capacity module 362 may use a timebanding model based on task deadlines, estimated work times, workflow contingencies, and other factors to project the cumulative workload and remaining capacity over a plurality of timebands. For example, work may be timebanded in 4-hour increments, 2-hour increments, 1-hour increments, etc. In some embodiments, cumulative capacity module 362 may be configured to simplify the cumulative capacity metrics into a single or time-series of capacity units used for capacity notification and workload management across the enterprise workload sharing system.

Capacity notifier 364 may use the worker availability calculated by cumulative capacity module 362 to inform other components of the processing store's current projected capacity. For example, capacity notifier 364 may generate periodic capacity notification messages to a work sharing coordinator, such as a shared work rules engine, directly to other stores, or published to a data store available to one or more other components. In some embodiments, capacity notifier 364 may use a capacity notification cadence or cycle time to determine how often cumulative capacity module 362 should update the cumulative capacity metrics based on changes in the local work queue 392 and/or other factors and send an updated capacity notification. In some embodiments, capacity notifier 364 may also monitor for a stop message from one or more other components to suspend sending capacity notifications.

Assigned work manager 370 may provide a user interface and related logic and data access to receive and complete remote work assignments. For example, assigned work manager 370 may allow computing system 300 to receive remote work assignment requests, integrate them into local work queue 392, enable a worker at the processing pharmacy (e.g., pharmacy resource) to complete the work task, and provide the disposition back to the source pharmacy. In some embodiments, assigned work manager 370 may include an assigned work dashboard 372, a work request handler 374, a work queue inserter 376, a work task display 378, a work task processor 380, and a status interface 382. In some embodiments, assigned work manager 370 integrates with existing pharmacy workflow systems, including local work manager 390, to provide backend enhancements and limited changes in work task display and processing to enable completion of remotely assigned work tasks.

Assigned work dashboard 372 may display assigned work tasks that are unrelated to locally sourced work, such as prescriptions received and being filled and delivered by the processing pharmacy. For example, assigned work dashboard 372 may display a list of prescription verification tasks and related parameters, such as transaction ID, patient, prescription, deadline, etc. In some embodiments, assigned work dashboard 372 may be integrated into a local work dashboard as an indicator or filter for identifying remotely assigned tasks and distinguish them from local tasks. In some embodiments, assigned work dashboard 372 may aggregate and report on work sharing metrics over a desired period of time, such as number of remote work tasks completed, and/or connect the assigned work metrics to overall store quotas or other metrics.

Work request handler 374 may receive assigned work requests from another system, such as a source pharmacy or a workload sharing coordinator. For example, work request handler 374 may receive an assigned work request message from a shared work rules engine, validate the request against one or more parameters, and return a status to the system sending the request, such as a success or failure message. In some embodiments, work request handler 374 may log the request to a local or central workshare log and pass the request and/or validated parameters to work queue inserter 376.

Work queue inserter 376 may inject one or more work tasks related to the received and validated work assignment into local work queue 392. For example, work queue inserter 376 may capture an induction time, such as the time the request was received, prioritize and sort the new task(s) against the local tasks already in local work queue 392, and set an action-by time. For example, the action-by deadline may be calculated by a buffer offset from a deadline set by the requester, such as a work delivery deadline or recall deadline.

Work task display 378 may provide a user interface for a worker, such as a pharmacist, to complete the assigned work task, such as prescription verification. For example, work task display 378 may use an existing prescription verification workflow for local prescription verification and enable it to process the assigned work task based on the received parameters and data access enabled by those parameters. Work task display 378 may display data related to the remote task, such as prescription information, related images, patient information, manufacturer information, reference tables and standards, etc. for conducting appropriateness review. Work task display 378 may provide navigation, prompts, rule-based guidance, and other elements of expert-guided data review and analysis. In some embodiments, one or more data source may not be provided within the parameters of the work request and/or not be available from local or enterprise data sources. For example, certain protected data resources, such as image files or other local data at the source pharmacy, may be desirable for completing the task. Work task display 378 may include an automated or user-selectable feature for accessing supporting data from the source pharmacy.

Work task processor 380 may receive and process a disposition for the completed task based on user input and/or internal processing logic. For example, work task display 378 may enable a pharmacist to select a disposition and enter any appropriate supporting information for the disposition. In some embodiments, work task processor 380 may capture additional information related to the disposition, such as supplemental directions for the source pharmacy or patient, changes to dosing schedule, reasons for rejection, clarification request, additional authorizations, etc. Work task processor 380 may validate the completeness of the disposition before initiating status interface 382.

Status interface 382 may provide status updates to one or more other systems, such as a source pharmacy, a shared work rules engine, or another shared work coordinator. For example, status interface 382 may format and send a work disposition message to the source pharmacy with the parameters necessary for the source pharmacy to complete the prescription fill. Status interface 382 may send a status message to a shared work coordinator, such as a shared work rules engine, and/or log the disposition as a shared work event. Status interface 382 may enable one or more other systems waiting for the disposition or expiry of the shared work assignment to complete their respective operations in response to receiving the status update.

Local work manager 390 may include one or more systems for managing work tasks at the processing pharmacy. For example, a processing pharmacy may have an existing pharmacy work flow application or pharmacy web portal used for managing the data entry, processing, data support, and various tasks related to filling a prescription. Local work queue 392 may include one or more work flows and accumulated tasks for a pharmacist and/or other staff to complete in support of the local work flows, such as receiving, processing, and filling prescriptions. Local work manager 390 and local work queue 392 may include logic and parameters for prioritizing, navigating, and selecting work tasks. In some embodiments, remotely assigned tasks may processed and integrated into local task queue 392 as if the prior step had been completed at the processing pharmacy and/or the follow-on steps could be completed at the processing pharmacy.

Pharmacy data source 398 may include data collected by and/or related to the processing pharmacy. For example, each pharmacy may collect and store retail and pharmacy operations data, such as point-of-sale data, supply chain data, pharmacy workflow data, employment data, etc. In some embodiments, pharmacy data source 398 may provide configuration and supporting data for processing pharmacy configuration 350, capacity manager 360, assigned work manager 370, and local work manager 390. In some embodiments, pharmacy data source 398 may support additional systems that may interact with shared work functions, such as a retail analytics system and/or workforce management system.

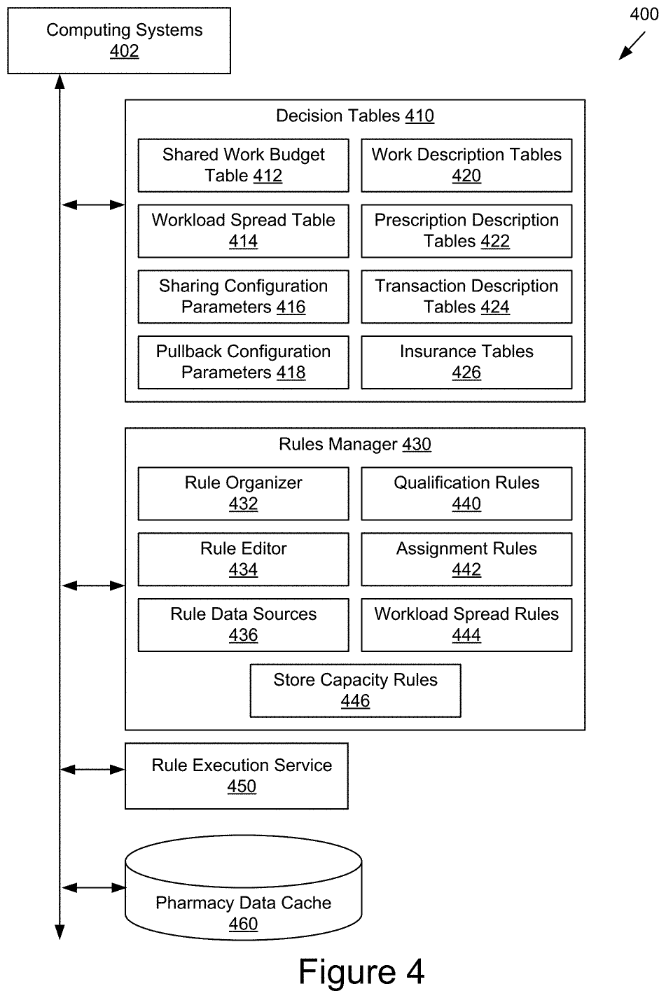

FIGS. 4-6 illustrate example enterprise-level components for a workload sharing platform 400, such as share work rules engine 130 and other components of enterprise service layer 104 in FIG. 1. In some embodiments, workload sharing platform 400 operates as a work share coordinator among source pharmacy computing systems, such as computing system 200 in FIG. 2, and processing pharmacy computing systems, such as computing system 300 in FIG. 3. The components of workload sharing platform 400 may be embodied and/or hosted on one or more computing systems 402. For example, workload sharing platform 400 may be operated from one or more enterprise data centers comprising scalable processing and storage systems and/or hosted solutions, such as through one or more cloud computing providers. In some embodiments, one or more of computing system 402 and their hardware/software components may be configured similar to computing system 200 and computing system 300 in FIGS. 2 and 3 respectively.

FIG. 4 shows an example decision tables module 410, rules manager 430, rule execution service 450, and pharmacy data cache 460. In some embodiments, a graphical user interface may be provided for enabling administrators of workload sharing platform 400 to navigate, review, update, and/or generate the contents of decision tables module 410 and rules manager 430. Decision tables module 410 may aggregate or connect to a plurality of data sources for supporting system configuration, rules manager 430, and other services of workload sharing platform 400. For example, decision tables module 410 may include a plurality of data tables or other data structures that may be easily navigated, updated, and/or dynamically created by administrators of workload sharing platform 400. In some embodiments, decision tables module 410 may include a shared work budget table 412, a workload spread table 414, sharing configuration parameters 416, pullback configuration parameters 418, work description tables 420, prescription description tables 422, transaction description tables 424, and insurance tables 426.

In some embodiments, shared work budget table 412 may include parameters to define how much work may be shared by each location, within various regions or other defined management hierarchies, or with regard to specific components or constraints within the system. Allocation of work share budgets may be managed based work tasks per time or another metric and at whatever level of granularity enables effective management of the allocations, such as daily, timebands, weekly, monthly, etc. For example, shared work budget table 412 may include entries for each store with work budget allocations defined on a daily basis and including both source and processing allocations.

In some embodiments, workload spread table 414 may provide parameters for spreading the workload from source pharmacies. For example, a table or other data structure may be used to define source workload spread per day and per hour. In some embodiments, a percentage source spread may be defined as a parameter. Sharing configuration parameters 416 may include one or more data structures for other sharing configuration parameters. For example, sharing configuration parameters 416 may include a table for defining which pharmacies are enabled for workload sharing on both the source and processing side, aggregating or extracting relevant pharmacy data into configuration parameters, parameter settings to enable or disable specific sharing features, capacity expiration rates, capacity notification cycle periods, etc. Pullback configuration parameters 418 may include one or more parameters in a data structure for defining one or more deadline calculations for managing work sharing. For example, pullback configuration parameters 418 may include various buffers, offsets, and/or algorithms for determining how much time will be allowed for processing a shared work request. Target work completion deadlines, automatic pullback deadlines, qualification and assignment deadlines, assignment confirmation deadlines, etc. may be calculated based on target processing times, back-off from one or more contingent tasks to meet a customer delivery deadline, and/or some combination thereof.

In some embodiments, decision tables module 410 may include a plurality of reference tables describing the range of parameters and relationships supported by workload sharing platform 400. For example, work description tables 420 may provide a list of work types subject to work share requests and/or parameter setting or ranges related thereto for validating work share requests and calculating capacity, deadlines, and other features specific to each work share type. Prescription description tables 422 may include one or more tables related to defining prescriptions and supporting decision-making, such as retail drug schedules, national drug codes, generic code numbers, etc. Transaction description tables 424 may include origin codes, prescriber types, prescription types, geographic or regulatory classifications, patient/customer types, etc. Insurance tables 426 may include data structures describing various insurers for prescriptions, such as third-party insurance plans, and may include prescription benefit and other details for a plurality of insurance plans.

Rules manager 430 may enable the administration of decision-making rules used by workload sharing platform 400. In some embodiments, the various services of workload sharing platform 400 use rule-based decision-making to evaluate and handle the parameters of work sharing messages and events. Rules manager 430 may aggregate the rule database used by the various services to more effectively manage contingencies, relationships, and supporting configurations and parameters, including the contents of decision tables module 410. In some embodiments, rules manager 430 may include rule organizer 432, rule editor 434, rule data sources 436 and a plurality of rule sets, such as qualification rules 440, assignment rules 442, workload spread rules 444, and store capacity rules 446.

In some embodiments, rules organizer 432 may provide a hierarchical and/or visual organizer for navigating the rule sets included in rules manager 430. For example, rules organizer 432 may include a graphical user interface for viewing, filtering, selecting, and otherwise navigating qualification rules 440, assignment rules 442, workload spread rules 444, and store capacity rules 446. In some embodiments, rules organizer 432 may enable a user to set rules order, nest rules, and/or assign effective dates to rules, such as temporary or updated rules. In some embodiments, rules editor 434 may provide an editor, such as a text editor or visual editor, for rules logic and/or parameters in rules manager 430. For example, rules editor 434 may enable a user of rules manager 430 to add, delete, or modify rules by selecting a rule entry or rule file. In some embodiments, rule data sources 436 may enable a user of rules manager 430 to access and manage one or more underlying data sources used by the rules sets. For example, rule data sources 436 may allow a user to access, define, update, or otherwise manage data sources used by qualification rules 440, assignment rules 442, workload spread rules 444, and store capacity rules 446, such as the parameters and tables in decision tables module 410 or pharmacy data cache 460.

In some embodiments, rules manager 430 may include a plurality of rule sets grouped by the service or services that may use them. For example, qualification rules 440 may include pre-qualification rules used by a source pharmacy computing system and/or qualification rules used by qualification service 530. Assignment rules 442 may include rules related to assigning work requests to processing pharmacies, such as used by assignment service 560. Workload spread rules 444 may include rules related to spreading work from one or more source pharmacies, such as used by qualification service 530. Store capacity rules 446 may include rules on how store capacity is modeled and acceptable loading parameters, such as used by assignment service 560.

Rule execution service 450 may be logic, scalable processor, and/or compute complex for evaluating rules sets in the context of specific events or work share transactions. For example, rule execution service 450 may be called by other services in workload sharing platform 400 to retrieve an up-to-date rule set and evaluate it based on the transactional parameters provided by the calling service. In some embodiments, rule execution service 450 may also support rule execution calls from other systems, such as the computing systems at source pharmacies or processing pharmacies.

In some embodiments, computing systems 402 may cache selected pharmacy data aggregated and stored by source and/or processing pharmacies to improve processing time and availability. For example, static pharmacy data, such as location, hours, services, etc., may be updated or synchronized periodically, but otherwise accessed from pharmacy data cache 460 by decision tables module 410, rules manager 430, and/or other services or components of workload sharing platform 400. In some embodiments, pharmacy data cache 460 may also receive and update capacity information from the processing pharmacies. For example, processing pharmacies may send periodic capacity updates that are stored in pharmacy data cache 460 for use by assignment service 560.

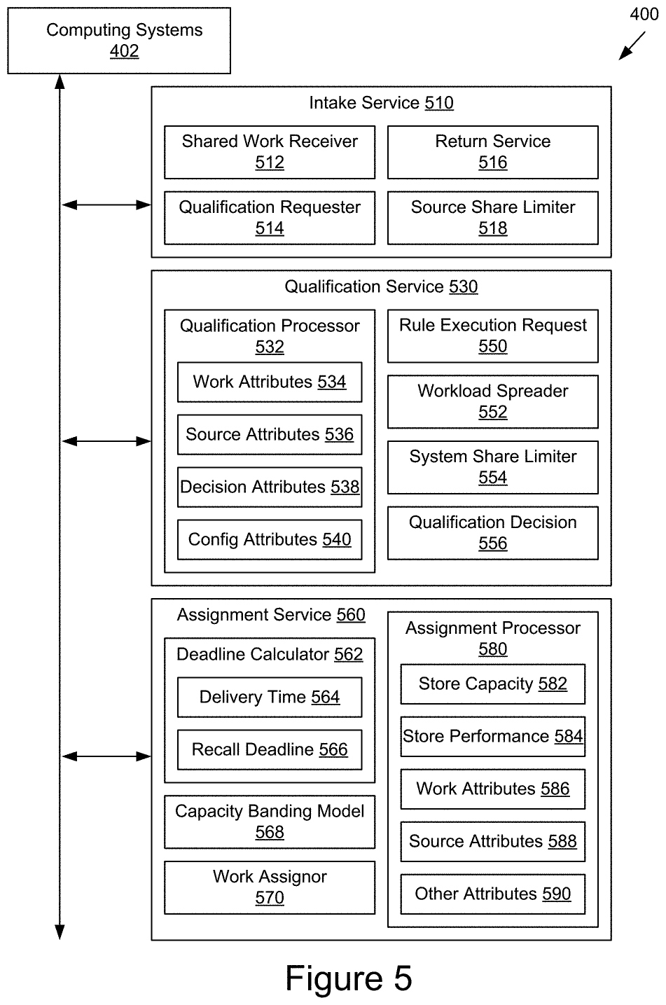

With regard to FIG. 5, intake service 510 may be configured to receive work share requests from source pharmacies and initiate processing by workload sharing platform 400. For example, intake service 510 may receive a work share request message from a source pharmacy, validate that the request parameters are complete and the source pharmacy is permitted to submit work share requests, and forward the request parameters to qualification service 530. In some embodiments, intake service 510 may include a shared work receiver 512, a qualification requester 514, a return service 516, and a source share limiter 518. For example, shared work receiver 512 may be a service for receiving work share requests from source pharmacies and may include logic for validating the form and content of the request for completeness. In some embodiments, shared work receiver 512 may also apply source share limiter 518 as a filter on the status, number, frequency, type, or other constraints for accepting work share requests from any given source pharmacy. For example, source share limiter 518 may track the daily load maximum allowed for each source pharmacy and set or receive a stop share indicator when that maximum is reached. If the shared work request is valid and under the source pharmacy's limits, qualification requester 514 may forward the work share request or parameter extracted therefrom to qualification service 530. Return service 516 may provide a return message to the requesting source pharmacy confirming receipt of the request. Return service 516 may deny or reject the request if it does not meet the parameter requirements or as directed by source share limiter 518. Return service 516 may otherwise indicate that the message has been forwarded for qualification and assignment. In some embodiments, intake service 510 may log requests and reply status, such as through logging interface 610. For example, logged requests may be used by source share limiter 518 to determine whether a new request is eligible for processing.

Qualification service 530 may be configured to qualify each work share request for assignment through workload sharing platform 400. For example, qualification service 530 may receive work share requests from intake service 510 and make qualification decisions. Qualified requests may be forwarded to assignment service 560 and unqualified or rejected requests may be returned to the source pharmacy for local processing. In some embodiments, qualification service 530 may include qualification processor 532, rule execution request 550, workload spreader 552, system share limiter 554, and qualification decision 556.

In some embodiments, qualification processor 532 may be configured to parse and/or retrieve attributes or parameters relevant to qualification rules for each shared work request received. For example, qualification processor may extract parameters from the shared work request to describe the work task, such as a prescription verification task with patient information, prescription information, source pharmacy information, and transaction information (e.g., transaction ID, customer delivery deadline, recall deadline, etc.). In some embodiments, qualification processor 532 may include work attributes 534 extracted from or related to the shared work request to describe the requested work task(s), such as prescription verification attributes. Qualification processor 532 may include source attributes 536 to describe the source pharmacy. In some embodiments, a source pharmacy ID may be associated with or included in the request and additional parameters may be retrieved from decision tables module 410 or pharmacy data, such as source pharmacy data in pharmacy data cache 460. Qualification processor 532 may include decision attributes 538 and configuration attributes 540 to index one or more decision tables and/or configuration parameters in decision tables 410.

Rule execution request 550 may format the relevant attributes into the parameters of a rule execution request, send it to rule execution service 450, and wait for a response qualifying or disqualifying the request based on the rules executed. In some embodiments, qualification processor 532 may parse a series of attribute sets corresponding to various rules sets and these rule sets will be executed using the attribute sets in a series of rule execution requests 550 to rule execution service 450. For example, qualification rules based on work attributes 534 may be sent to rule execution service 450. If passed, qualification rules based on source attributes 536 may be sent to rule execution service 450, and so on.

In some embodiments, qualification service 530 may include workload spreader 552 and system share limiter 554 to balance source pharmacy work share requests at a system level. For example, workload spreader 552 may track incoming requests and determine whether an otherwise qualifying request should be rejected due to overuse of the system by a particular source, region, time, or some other characterization. System share limiter 554 may further evaluate share traffic against system or enterprise-wide metrics and quotas for use of workload sharing. System share limiter 554 may include an indicator or control a configuration setting for enabling or disabling intake and/or qualification of work share requests based on system quotas being met enterprise-wide or at regional or other organizational levels for a given period, such as timeband, day, week, etc.

Qualification decision 556 may be triggered when all qualification rules relevant to the request have been processed through qualification processor 532 and rule execution requests 550. In some embodiments, qualification decision 556 may also require passing additional filters, such as workload spreader 552 and system share limiter 554. Qualification decision 556 may communicate the qualification decision and/or parameters for further processing to assignment service 560. In some embodiments, qualification decision 556 may also log the qualification decision and/or provide a qualification status message back to the source pharmacy.

Assignment service 560 may assign each qualified work share request to a processing pharmacy in accordance with one or more assignment rules. For example, assignment service 560 may be called by or in response to a qualification decision by qualification service 530. In some embodiments, assignment service 560 may include a deadline calculator 562, a capacity banding model 568, a work assignor 570, and an assignment processor 580.

Deadline calculator 562 may prepare for assignment processing by determining one or more deadlines for work tasks, which may be used to determine which processing pharmacies are eligible for assignment (i.e., have capacity to complete the tasks by the time it needs to be completed). For example, deadline calculator 562 may receive a customer delivery time for a prescription as a parameter of the work share request and calculate one or more deadlines related to completion of the remote work task by the processing pharmacy. In some embodiments, deadline calculator 562 may include a work delivery time calculator 564 and a recall deadline calculator 566. For example, work delivery time calculator 564 may apply production completion buffer for the source pharmacy to the customer delivery deadline to calculate a work delivery time or processing promise time for delivery of the disposition back to the source pharmacy. In some embodiments, the work delivery time may also be calculated based on work processing time and average work queue depths and the earlier of the work time calculation or the completion buffer may be used as the work delivery time. Recall deadline calculator 566 may calculate a second deadline for automatically recalling the work task and providing it back to the source pharmacy. In some embodiments, recall deadline calculator 566 may include both a work completion buffer and a work task time, assuming that the source pharmacy will need the time to complete both the requested task and any contingent tasks, such as production completion and filling. In some embodiments, deadline values from deadline calculator 562 may be provided as parameters to assignment processor 580.

Capacity banding model 568 may provide a time-based banding model for placing the work request in an appropriate projected timeband for assignment and completion. For example, capacity banding model 568 may receive periodic capacity updates from processing pharmacies regarding their capacity and work queues projected into future timebands. In some embodiments, capacity banding model 568 may load real-time or near real-time capacity information received from all available processing pharmacies, such as capacity data stored in pharmacy data cache 460. For example, capacity banding model 568 may assemble the capacity data for all eligible processing pharmacies into a table, array, or other data structure for providing current capacity projected across timebands for all processing pharmacies under consideration.

Work assignor 570 may be responsible for communicating the work share assignment to the assigned processing pharmacy selected through assignment processor 580. For example, if a processing pharmacy is successfully assigned by assignment processor 580, work assignor 570 may populate and send an assigned work request message to the computing system of the processing pharmacy assigned. In some embodiments, work assignor 570 may also log the assignment action and/or communicate the assignment status to the source pharmacy. In the event that no processing pharmacy meeting the assignment parameters can be found for the shared work request and/or the assigned processing pharmacy denies the request, non-assignment and termination of the request may be logged and communicated to the source pharmacy. In some embodiments, work assignor 570 may also receive assignment confirmation messages from processing pharmacies in response to the work share assignment message. For example, work assignor 570 may receive a success/failure or accept/reject message from the processing pharmacy in response to the work share assignment message.

Assignment processor 580 may be configured to parse and/or retrieve attributes or parameters relevant to assignment rules for each shared work request received. For example, assignment processor 580 may extract parameters from the shared work request to describe the work task, such as a prescription verification task with patient information, prescription information, source pharmacy information, and transaction information, including deadlines calculated by deadline calculator 562. Assignment processor 580 may use capacity banding model 568 to generate store capacity 582 for each processing store being considered for assignment. Other attributes used by assignment processor 580 may include store performance attributes 584, work attributes 586, source attributes 588, and other attributes 590, such as decision and configuration attributes from decision tables module 410.

In some embodiments, assignment processor 580 may process one or more sets of attributes against an initial set of processing pharmacy candidates to filter ineligible processing pharmacies. For example, assignment processor 580 may submit attributes to rule execution service 450 to identify regional restrictions, licensing or insurance incompatibilities, and similar features that would disqualify one or more processing pharmacies, such as incompatible state laws. Similarly, performance-based rules, basic available capacity or quota restrictions, and other characteristics may enable processing pharmacies to be filtered prior to calculation of an assignment from among a filtered set of eligible processing pharmacies. Assignment processor 580 may also receive a capacity rating for processing pharmacies and/or capacity distribution across a set of processing pharmacies from capacity service 602 for use in generating assignments. In some embodiments, assignment processor may query a capacity database maintained by capacity service 602 and select the highest scored store meeting its search criteria, such as geographic restrictions.

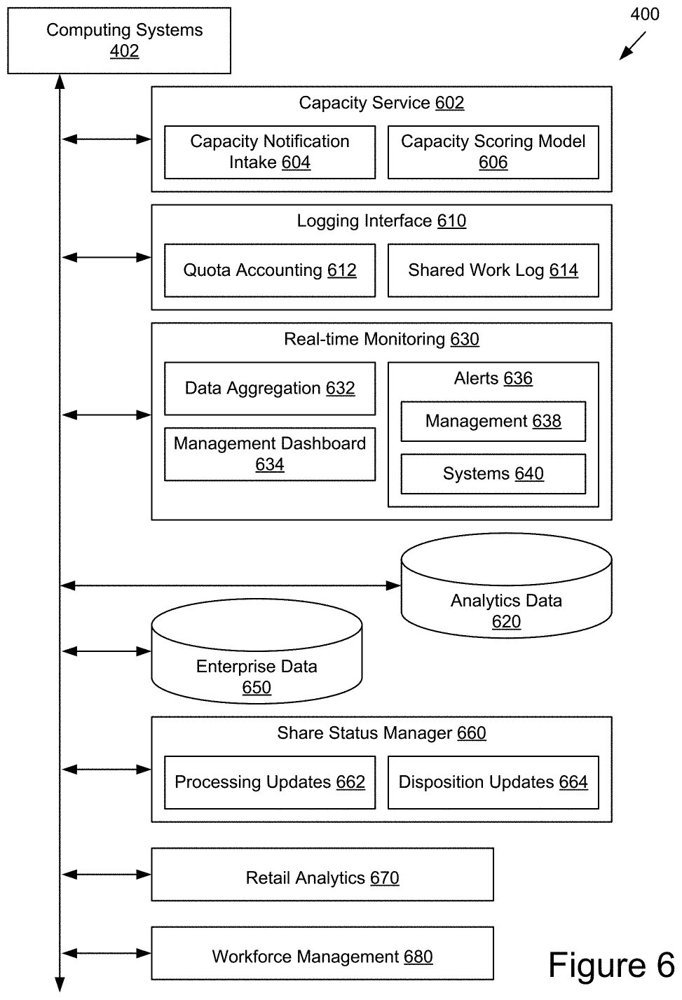

With regard to FIG. 6, capacity service 602 may aggregate all relevant attributes for evaluating capacity for use in an assignment decision. Relevant capacity data may include static data regarding various processing pharmacies, as well as dynamic capacity information received through capacity notification intake 604. Capacity data may be processed through capacity scoring model 606 for making weighted assignment decisions. Capacity service 602 may provide capacity scoring information to assignment processor 580 and/or other components of workload sharing platform 400 for making assignment decisions.

In some embodiments, capacity notification intake 604 receives periodic capacity notifications from processing pharmacies. For example, a selected pharmacy may enable shared work processing or it may be enabled by workload sharing platform 400 and may be in an active state for new processing tasks (e.g., has not received a stop order). Each such pharmacy may send a periodic capacity notification on a capacity sharing cadence, such as every 5 minutes. Capacity notification intake 604 may receive and store these notifications. For example, capacity notification intake 604 may include a capacity data table that includes: static data, such as pharmacy identifier, hours, and daily quota; calculated data, such as time remaining in day, assignment counter, percentage of day available, percentage of quota available; and notification data, such as projected workload or capacity available, which may be distributed across timebands. Capacity notification intake 605 may periodically call capacity scoring model 606 to recalculate capacity scoring based on updated capacity notifications.

In some embodiments, capacity scoring model 606 may include calculating an assignment score for each possible processing pharmacy for use in assigning the processing pharmacy. In some embodiments, the assignment score alone may be sufficient to make an assignment selection (e.g., highest assignment score is assigned) or all possible processing pharmacies within a score band may be candidates for selection algorithm, such as round robin. In some embodiments, capacity scoring model 606 may use capacity banding model 568 for each processing pharmacy and calculate assignment scores for one or more eligible time bands relevant to the time constraints of the work share request. In some embodiments, capacity scoring model 606 may be instantiated through a set of capacity evaluation rules executed through rules execution service 450. For example, capacity evaluation rules may include thresholds, such as: maximum capacity available (in seconds) over which a processing pharmacy has the highest possible capacity rating in a given time band; and minimum capacity available (in seconds) under which a processing pharmacy has the lowest possible capacity rating in a given time band.

In some embodiments, capacity scoring model 606 may include a capacity rating (in terms of available worker-time) and a quota rating (in terms of amount of quota remaining to be filled) to determine the assignment score value for each processing pharmacy being considered. A configurable weighting value may be used for weighting the remaining quota and/or the available capacity. In some embodiments, individual timeband capacity scores may be multiplied by the minimum cumulative capacity time band score to rate the timeband capacity. An example assignment score calculation may be (Weightquota*% Quota Remaining)+(Individual Time Band Capacity Score*minimum (Cumulative Capacity Time Band Scores)). In some embodiments, the quota remaining may be recalculated after each successful assignment and capacity scores may be recalculated after each capacity notification is received from the processing pharmacies, such as every 4 minutes or another capacity update cycle.

Logging interface 610 may receive log entries for shared work actions or events from other components or services of workload sharing platform 400. For example, logging interface 610 may provide access to one or more data structures for quota accounting 612, such as storing counts of shared work against one or more quotas at the source/processing pharmacy level and/or various aggregate levels for the regions, systems, etc. A running total of shared work per source and processing pharmacy may be generated from quota accounting 612. In some embodiments, quota accounting 612 may log work share requests completed and track them against store identifiers. For example, quota accounting 612 may provide the shared work counts for storage in analytics data store 620 alongside other pharmacy performance data. Quota and other analytics data may be aggregated and analyzed for setting and revising quotas or taking other management actions. Shared work log 614 may accumulate log entries for each action or event related to the processing of shared work transactions. In some embodiments, shared work log 614 includes timestamped entries corresponding to actions and events along the work share process. Shared work log 614 may be stored, aggregated, and/or analyzed within analytics data store 620 to manage the operation of workload sharing platform 400 and/or the aggregate actions of specific source and/or processing pharmacies and/or other work request parameters.

Real-time monitoring module 630 may enable aggregate activity through workload sharing platform 400. For example, real-time monitoring module 630 may collect transaction and log data, store some or all of the collected data within enterprise data store 650 and enable data analytics and management access to such data. In some embodiments, real-time monitoring module 630 may include data aggregation module 632 for collecting data related to operation of workload sharing platform 400, such as quota accounting 612 and shared work log 614. For example, data aggregation module 632 may collect all transactional, event, log, and/or session data passing through workload sharing platform 400 over fixed time segments, such as 24-hour periods, and archive and/or abstract and store defined metrics and/or queries. In some embodiments, real-time monitoring module 630 may include management dashboard 634 for providing summary results and/or pre-built views or queries based on aggregated data. For example, management dashboard 634 may include data mining interfaces and data connections to one or more pre-existing business intelligence systems or enterprise management dashboards. In some embodiments, real-time monitoring module 630 may include automated alerts 636 based on the aggregate data, such as management alerts 638, related to business metrics, and/or system alerts 640, related to information technology operational metrics. For example, real-time monitoring module 630 may maintain ongoing metric collection and/or periodic queries against the aggregate data which are compared against alert thresholds or trigger values with corresponding automated response. For example, when the number, type, source, or processing transactions exceed a statistical norm, alerts may be sent to appropriate business managers and/or system administrators.

Share status manager 660 may provide an intermediary for handling status notifications from one or more other components. For example, share status manager 660 may monitor for log entries or status messages from processing pharmacy computing systems and propagate those status changes to other components. In some embodiments, share status manager 660 may receive processing updates 662 and/or disposition updates 664 from processing pharmacy computing systems. These updates may be formatted as log entries and sent to logging interface 610. In some embodiments, processing updates 662 and/or disposition updates 664 may be provided to source pharmacy computing systems or other system components to respond to the status change or disposition.

Workload sharing platform 400 may interact with a variety of other enterprise layer systems, such as retail analytics systems 670 and workforce management systems 680. For example, retail analytics systems 670 may include one or more data analytics, management, and/or planning tools that track and analyze retail sales, customer, and supply chain data to inform purchasing, promotion, management, and other decisions, often utilizing enterprise data store 650. In some embodiments, retail analytics systems 670 may be used to set quotas, improve capacity prediction algorithms, capacity banding algorithms, and assignment scoring models. Workforce management systems 680 may include one or more data analytics, management, and/or planning tools that track and analyze human resource and performance data to inform staffing, hiring, compensation, capacity planning, and other decisions, often utilizing enterprise data store 650. In some embodiments, workforce management systems 680 may be used to generate and update task and productivity tables for use in decision tables module 410 and performance against quotas and processing times may contribute to workforce productivity and compensation calculations.

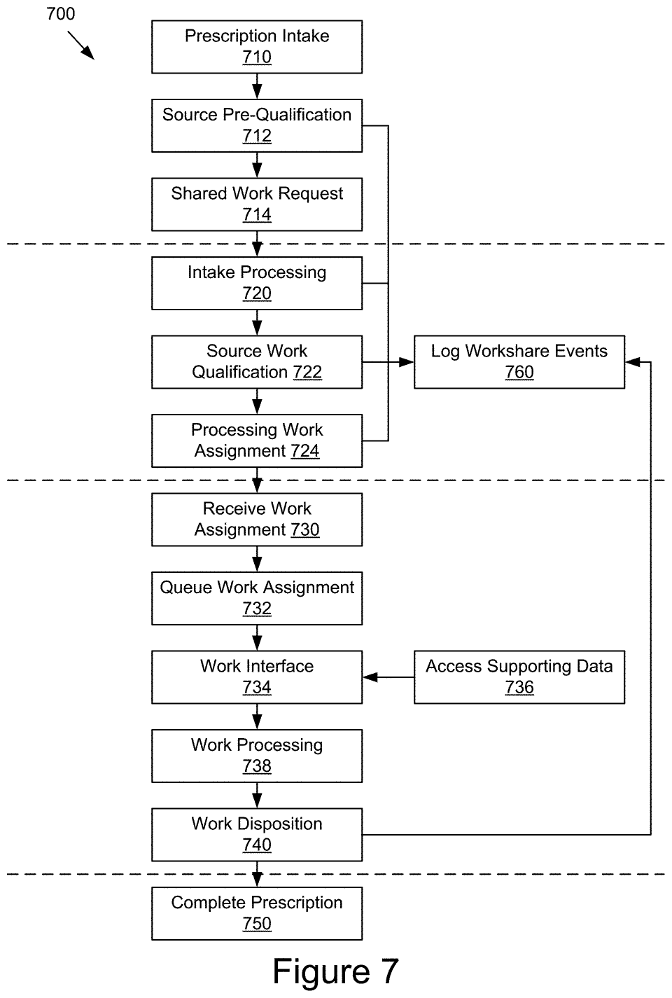

FIG. 7 shows an example method 700 of workload sharing using an enterprise workload sharing platform and associated pharmacy computing systems. For example, method 700 may enable a source pharmacy computing system, such as computing system 200 in FIG. 2, to provide a remote work assignment to a processing pharmacy computing system, such as computing system 300 in FIG. 3, through a work sharing coordinator, such as workload sharing platform 400 in FIGS. 4-6.

At block 710, prescription intake may be completed, such as at a source pharmacy. For example, a prescription may be received from a patient, prescriber, or medical information system for filling at the source pharmacy. At block 712, the source pharmacy may pre-qualify that prescription for work sharing. For example, the source pharmacy may have the necessary sharing resources enabled and not be subject to any exceeded quotas or restrictions of work sharing. At block 714, a shared work request may be generated for the prescription. For example, a shared work request message with parameters describing the prescription verification task to be completed may be sent to a work sharing coordinator. In some embodiments, blocks 710, 712, and 714 may be executed by a computing system at or accessible from the source pharmacy.

At block 720, an intake process for the shared work request may be executed by the work sharing coordinator in response to the shared work request. For example, the shared work request may be parsed for valid parameters and source pharmacy before passing the shared work request to qualification and assignment engines. At block 722, the shared work request and source pharmacy may be evaluated against one or more qualification rules to determine whether the shared work request is a candidate for assignment to a processing pharmacy. For example, parameters from the shared work request and related source pharmacy parameters may be evaluated against a plurality of qualification rules to qualify for shared work assignment. At block 724, the shared work request may be evaluated against the available processing pharmacies to determine whether there is a pharmacy capable of processing the request and, if so, which one. For example, a set of possible processing pharmacies may be identified based on available capacity and other factors and compared against the processing deadline and other parameters of the shared work request to select a processing pharmacy for assignment and send a shared work assignment request to the assigned processing pharmacy. In some embodiments, blocks 720, 722, and 724 may be executed by an enterprise service layer hosted remotely from the source pharmacy and the processing pharmacy.

At block 730, the shared work assignment may be received by the processing pharmacy. For example, the shared work assignment request may be received and validated against the available capacity, parameters, and deadline for completing the shared work assignment. At block 732, the shared work assignment may be queued alongside local work tasks at the processing pharmacy. For example, queuing logic for prioritizing tasks for completion by staff, such as pharmacists, may place the shared work task(s) based on submission time, completion deadline, and other factors used for queuing local work tasks. At block 734, a pharmacy resource may use a work interface to complete the shared work assignment and select a disposition for the request. For example, the pharmacist may select a remote prescription verification task from the task queue and use the pharmacy workflow interface to complete the task as if it were associated with a local prescription, selecting a disposition and providing any expert input necessary to support subsequent tasks from the disposition. At block 736, the pharmacy resource may access supporting data from the source pharmacy. For example, the pharmacist may access image files or database information related to the patient, prescription, or production process at the source pharmacy. At block 738, one or more tasks associated with the shared work assignment may be processed at the processing pharmacy. For example, the pharmacist's inputs at block 734 may complete the workflow, close the shared work assignment, and enable the processing pharmacy computing system to complete any remaining processing of the parameters and inputs to support the disposition. At block 740, the shared work disposition may be generated and sent to the source pharmacy. For example, a disposition message including parameters to support any further processing and/or production of the prescription in compliance with the prescription verification may be sent to the source pharmacy and a status update may be provided to other systems, such as the shared work coordinator. In some embodiments, blocks 730, 732, 734, 736, 738, and 740 may be executed by a computing system at or accessible from a processing pharmacy.

At block 750, the prescription may be completed at the source pharmacy in response to receiving the disposition from the processing pharmacy. For example, production verification and filling tasks for the prescription may be placed in the local work queue at the source pharmacy to complete filling for the prescription. In some embodiments, at block 760, workshare events may be logged in response to activities at various blocks, such as blocks 712, 720, 722, 724, and 740. For example, each block may generate one or more shared work log entries stored by the shared work coordinator.

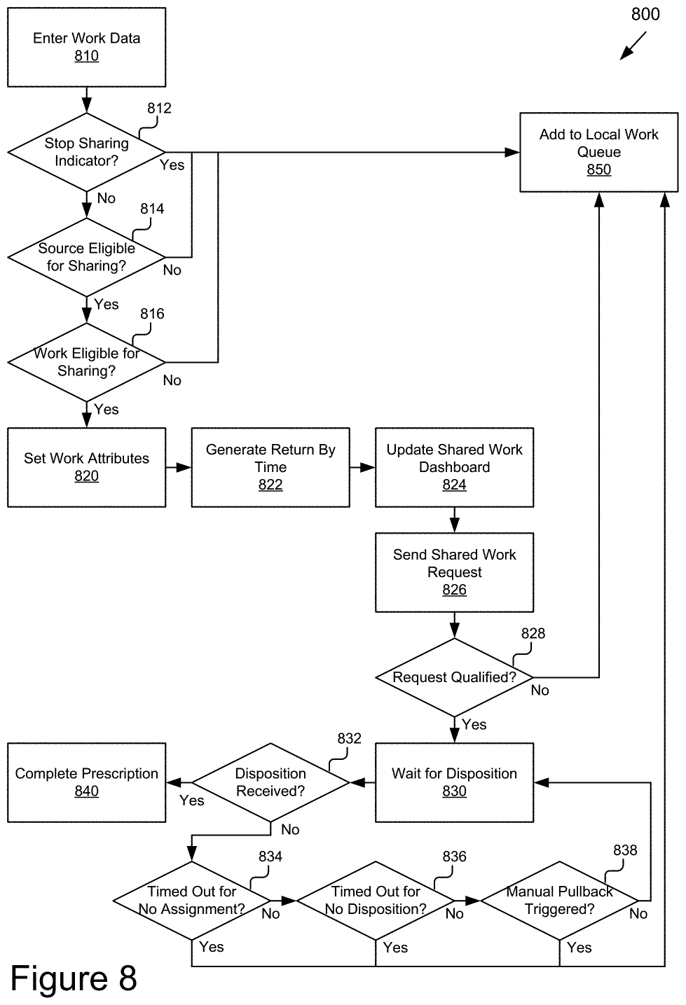

FIG. 8 shows an example method 800 for sharing work tasks from a source location, such as a source pharmacy. For example, method 800 may enable a source pharmacy computing system, such as computing system 200 in FIG. 2, to provide a remote work request to a processing pharmacy computing system, such as computing system 300 in FIG. 3, through a work sharing coordinator, such as workload sharing platform 400 in FIGS. 4-6, and receive a disposition of the shared work request to enable completion of a contingent work flow, such as processing and filling a prescription.

At block 810, work data may be entered into or otherwise received by a computing system. For example, a prescription may be received and relevant parameters identified from an electronic data exchange format and/or entered at the source pharmacy. At block 812, the computing system may check for a stop sharing indicator to determine whether work sharing is currently enabled for the source pharmacy. If the stop sharing indicator indicates yes (e.g., a work sharing quota has already been reached), then work sharing is currently disabled and the received work may be processed locally through a local work queue at block 850. Otherwise, method 800 may continue to block 814. At block 814, the computing system may determine whether the source location, such as the source pharmacy, is eligible for sharing work. If the source location is not eligible for work sharing (e.g., the source location has insufficient work in queue), then the received work may be processed locally through a local work queue at block 850. If the source location is eligible for work sharing, method 800 may continue to block 816. At block 816, the computing system may determine whether the work task, such as prescription verification for a specific prescription and patient, is eligible for work sharing. If the work task is not eligible for work sharing (e.g., the patient or prescription match work sharing exclusion criteria), then the received work may be processed locally through a local work queue at block 850. If the work task is eligible for work sharing, method 800 may continue to block 820.

At block 820, the computing system may parse and set the work attributes for the work task to be completed. For example, a transaction ID may be associated with a set of parameters describing the prescription, patient, and other information relevant to completing the work task remotely. At block 822, the computing system may generate a return-by time or work completion deadline for the shared work task. For example, there may be a customer delivery deadline defined for the prescription and the computing system may calculate the time required to complete the remaining tasks to fill the prescription after the remote verification is complete, with or without additional margin for local work queue or other factors. At block 824, the computing system may update a shared work dashboard to reflect the new shared work request. For example, a new entry for the prescription may be added to the list of shared work viewable in the shared work dashboard and/or from the patient or prescription records in a pharmacy management application. In some embodiments, the prescription and shared work may not appear in the local work queue at this stage. At block 826, the shared work request and related parameters may be sent to a shared work coordinator, such as workload sharing platform 400 or shared work rules engine 130. For example, the computing system may format a shared work request message addressed to a shared work request handler in the enterprise service layer supporting the computing system. At block 828, the computing system may receive a determination of whether the request has been received and/or qualified for shared work assignment. For example, the shared work coordinator may provide a response message to the shared work request message that may acknowledge receipt and/or provide status information regarding qualification of the request and/or assignment to a processing location. If the request did not qualify (e.g., source quotas reached, source load balancing, lack of processing capacity, etc.), then the request may be canceled and the received work may be processed locally through a local work queue at block 850. If the request is qualified for work sharing, method 800 may continue to block 830.

At block 830, the computing system may wait for disposition and/or cancelation of the request. For example, the computing system may include a status handler that receives status and/or disposition messages from the shared work coordinator and/or processing location. At block 832, the computing system checks whether a disposition has been received. If a disposition has been received, method 800 may continue to completing the work flow, such as filling (or denying) the prescription, at block 840. If the disposition has not been received, method 800 may continue to block 834. At block 834, the computing system evaluates whether the request should be timed out for no assignment. In some embodiments, the shared work coordinator may provide a status message when the request is assigned to a processing pharmacy and the computing system may include an assignment deadline by which the status message must be received of the request should be canceled. If the assignment deadline has been reached, then the request may be canceled and the received work may be processed locally through a local work queue at block 850. If the deadline has not been reached or an assignment message has been received, method 800 may continue to block 836. At block 836, the computing system evaluates whether the request should be timed out for no disposition. If the pullback deadline has been reached, then the request may be canceled and the received work may be processed locally through a local work queue at block 850. If the pullback deadline has not been reached, method 800 may continue to block 838. At block 838, the computing system evaluates whether a manual pullback has been triggered. For example, the pharmacy workflow application may include a selection from the shared work dashboard, prescription record, and/or patient record to manually cancel the shared work request. If a manual pullback has been triggered, then the request may be canceled and the received work may be processed locally through a local work queue at block 850. If the manual pullback has not been triggered, method 800 may return to block 830.

At block 840, a disposition has been received at block 832. For example, a disposition message has been received from the shared work coordinator and/or processing pharmacy that includes parameters describing the disposition and any supporting data. Based on the disposition type, the computing system may generate additional work tasks for completing or denying the prescription based on the remote prescription verification.

At block 850, the received work has not been completed remotely and is added to the local work queue. For example, the prescription may be processed according to local work flow without regard to remote work sharing. In some embodiments and cases, the local work may need to be prioritized or handled with rush status due to time elapsed during one or more remote work sharing attempts.

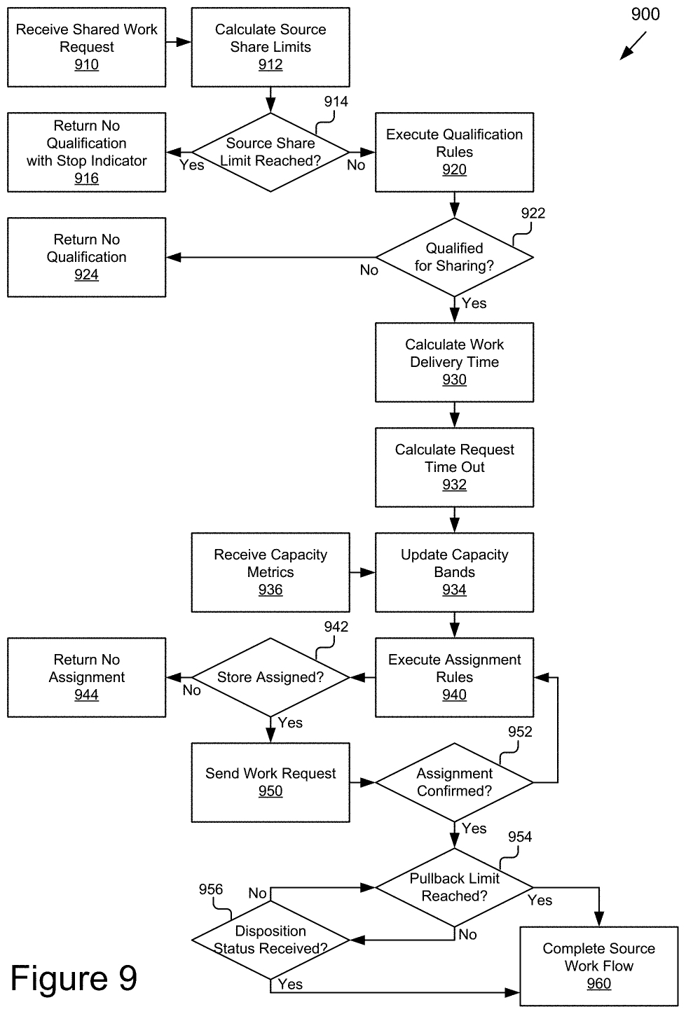

FIG. 9 shows an example method 900 for coordinating shared work tasks from a source location, such as a source pharmacy, to a processing location, such as a processing pharmacy. For example, method 900 may enable a work sharing coordinator, such as workload sharing platform 400 in FIGS. 4-6, to receive a shared work request from a source pharmacy computing system, such as computing system 200 in FIG. 2, and provide a remote work assignment to a processing pharmacy computing system, such as computing system 300 in FIG. 3.

At block 910, a computing system may receive a shared work request. For example, a computing system in an enterprise services layer may receive a shared work request message from a source pharmacy. At block 912, the computing system may calculate source sharing limits. For example, each source pharmacy may have a quota of work sharing they are allowed during any given time period and/or sharing limits may be dynamically calculated to balance shared work loads among source pharmacies. At block 914, the computing system may evaluate whether one or more source sharing limits have been reached. If a limit has been reached, then method 900 may proceed to block 916. If no limit has been reached, then method 900 may proceed to block 920. At block 916, the computing system may return a no qualification message to the source location and may include a stop indicator. For example, the computing system may send a no qualification message with a stop indicator to the source pharmacy when the quota limit has been met and the source pharmacy may suspend further shared work requests until the stop indicator expires or a start indicator us received.

At block 920, the computing system may execute qualification rules based on parameters received or derived from the shared work request. For example, the computing system may parse parameters corresponding to one or more rule sets, such as evaluations of work attributes, source attributes, etc., and process them through a rules execution engine to generate a qualification decision. At block 922, the computing system may evaluate whether the shared work request is qualified for sharing. If the shared work request does not pass all of the share qualification rules, then method 900 may proceed to block 924. If the shared work request does pass all of the share qualification rules, then method 900 may proceed to block 930. At block 924, the computing system may return a no qualification message to the source location.

At block 930, the computing system may calculate a work delivery time. For example, a work delivery time for the work task or tasks in the request may be based on a customer delivery deadline or a task completion deadline assigned by the source location. At block 932, the computing system may calculate a request time out or pullback deadline for canceling any assigned work and returning it to the source pharmacy for completion. For example, the request time out may be based on an estimated task completion time with or without additional buffers for returning the work to the source pharmacy. At block 934, the computing system may update capacity timebands for all processing locations in the pool of possible recipients of the shared work request. For example, the computing system may calculate both aggregate capacity across locations and an individual capacity metric in each timeband that may be relevant to assigning the work request. In some embodiments, capacity metrics may be received from each processing location at block 936. For example, each processing pharmacy may send a periodic capacity update based on their actual and/or projected work queue on a defined cycle, such as every 4 minutes.

At block 940, the computing system may execute assignment rules to select a processing location for the shared work request. For example, the computing system may use parameters of the work request and potential processing pharmacies to eliminate processing pharmacies that lack capacity or credentials for completing the work task and the submit the capacity banding models for the remaining processing pharmacies to be processed according to a work assignment scoring model to score and select a processing pharmacy. At block 942, the computing system may evaluate whether a processing location has been assigned. If a processing location is assigned, then method 900 may proceed to block 950. If no processing location is assigned (e.g., all possible processing pharmacies excluded by one or more assignment rules and/or do not meet a scoring threshold for assignment), the method 900 may proceed to block 944. At block 944, the computing system may return a no assignment message to the source location.

At block 950, the computing system may send an assigned work request to the assigned processing location. For example, the computing system may generate a shared work assignment request to the processing pharmacy identified at block 940 that contains parameters for completing the work task, such as a prescription verification. At block 952, the computing system may evaluate whether the assignment has been confirmed by the processing location. For example, the computing system may wait for an assignment confirmation message in response to the shared work assignment request. If the assignment is confirmed, method 900 may proceed to block 954. If the assignment is not confirmed, method 900 may return to block 940 to determine whether another processing location is available. At block 954, the computing system may evaluate whether a pullback limit or deadline has been reached. If the time for completing the assigned request has not been reached, then method 900 may proceed to block 956. If the time for completing the assigned request has been reached, then method 900 may proceed to block 960. At block 956, the computing system may evaluate whether the disposition status for the assigned shared work request has been received from the processing location. If the disposition status has not been received, method 900 may return to block 954 to continue to evaluate the elapsed time for completing the request. If the disposition status has been received, then method 900 may proceed to block 960.

At block 960, the computing system may terminate the shared work request and enable the source location to complete any remaining workflow steps. For example, if the shared work request has been completed, the disposition and any related parameters may be provided to the source pharmacy and remaining or contingent tasks from the disposition may be handled through the local work queue at the source pharmacy. If the shared work request has not been completed (e.g., no disposition received before pullback limit, work assignment terminated by processing or source pharmacy before completion, etc.), then the shared work task(s) may be returned to the source pharmacy and added to the local work queue.

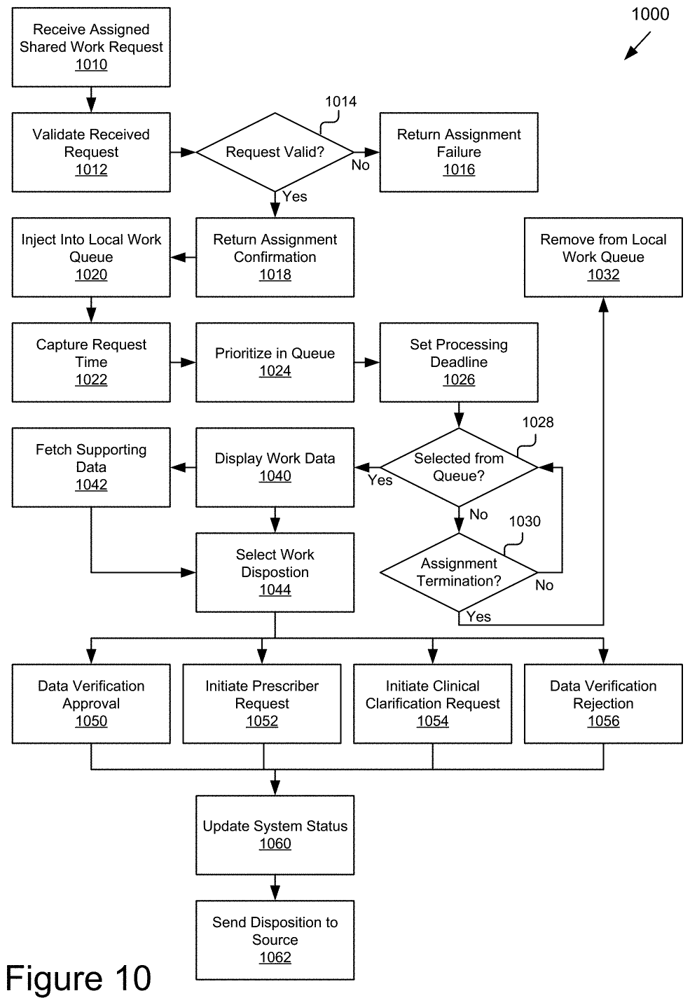

FIG. 10 shows an example method 1000 for processing shared work tasks at a processing location, such as a processing pharmacy, that is in a geographically different location than a source location, such as a source pharmacy. For example, method 1000 may enable a processing pharmacy computing system, such as computing system 300 in FIG. 3, to receive a shared work request from a source pharmacy computing system, such as computing system 200 in FIG. 2, through a work sharing coordinator, such as workload sharing platform 400 in FIGS. 4-6.

At block 1010, a computing system may receive an assigned shared work request. For example, a work sharing coordinator may assign the shared work request to the processing pharmacy and send an assigned shared work request message to the computing system. At block 1012, the computing system may validate the received shared work request. For example, the computing system may validate that the parameters necessary for completing the requested task(s) are included with the request and that the capacity of the processing pharmacy is still sufficient to complete the task within the time requested. At block 1014, the computing system may determine whether the request was successfully validated. If the request failed to validate, then method 1000 may proceed to block 1016. If the request was successfully validated, then method 1000 may proceed to block 1018. At block 1016, the computing system may return an assignment failure status message to the work sharing coordinator and/or source location. At block 1018, the computing system may return an assignment confirmation status message to the work sharing coordinator and/or source location.

At block 1020, the computing system may inject one or more work tasks based on the shared work assignment into a local work queue. For example, a shared work assignment for prescription verification may generate a prescription verification task for addition to the local work queue used for processing prescriptions at the processing pharmacy. At block 1022, the request time is captured. For example, the request time may be set to the time of receipt of the assignment message or based on the original prescription request time provided in the parameters of the request. At block 1024, the computing system may prioritize the work tasks in the local work queue, including the shared work assignment. For example, the order of the local work queue may be reordered to integrate the shared work task in a position appropriate to its request time and completion deadline, such as using the prioritization algorithm present in the workflow system managing the local work queue. At block 1026, the computing system may calculate and set a local processing deadline. For example, the request may include a completion deadline based on a customer delivery deadline with appropriate setback or buffer values for completing the remaining work tasks at the source pharmacy and the computing system may calculate the local processing deadline based on the available capacity and projected workload based on the local work queue and/or local workload patterns. At block 1028, the computing system may present the local work queue for task selection at the processing location, such as by a pharmacist. If the shared work task is not selected from the queue, then method 1000 may proceed to 1030. If the shared work task is selected from the queue, then method 1000 may proceed to block 1040.

At block 1030, the computing system may check for termination of the shared work assignment. For example, the computing system may monitor one or more completion deadlines that may trigger termination of the assignment and/or monitor for termination messages from the shared work coordinator and/or source pharmacy. If an assignment termination is not triggered, then method 1000 may return to block 1028 to monitor for selection of the shared work task for completion by the pharmacy resource. If an assignment termination is triggered, then method 1000 may proceed to block 1032. At block 1032, the computing system may remove the shared work tasks from the local work queue and terminate the shared work assignment.

At block 1040, the computing system displays work data for completion of a work task from the shared work request. For example, a prescription verification task may be displayed through a pharmacy workflow application for completion of prescription appropriateness verification be a pharmacist. At block 1042, the computing system may fetch remotely located work data for completion of the work task. For example, supporting data may be automatically or selectively retrieved from one or more remote data sources, such as a data source at the source pharmacy containing image files or other data not available locally. At block 1044, the computing system may select a disposition for the work task. For example, the computing system may provide a list of one or more dispositions which may be selected and/or verified by an expert user, such as a pharmacist, based on the data provided and supporting work flow logic.

Blocks 1050, 1052, 1054, and 1056 provide example dispositions that may be selected through the computing system in some embodiments of the invention. At block 1050, data verification approval may be selected as a disposition. For example, the pharmacist may complete the necessary series of verification steps and indicate completion and approval of the prescription as submitted or with permitted modifications. At block 1052, initiate prescriber request may be selected as a disposition. For example, the pharmacist, with the assistance of the computing system, may identify questions or inconsistencies between the prescription, patient information, and related guidance on appropriateness, insurance coverage, cost, etc. that should be resolved by the prescriber prior to filling the prescription. At block 1054, initiate clinical clarification request may be selected as a disposition. For example, the pharmacist, with the assistance of the computing system, may identify questions or additional instructions for the patient that should be resolved by the source pharmacy prior to or in association with filling and delivering the prescription to the patient. At block 1056, data verification rejection may be selected as a disposition. For example, the prescription and related information may include errors or inconsistencies that may not be resolvable through clarifications and the prescription should be denied.

At block 1060, the computing system may update the status of the assigned shared work request. For example, the computing system may send a status update message to the source pharmacy and/or work share coordinator. The shared work task status may also be updated within the local work queue and removed such that it does not generate the follow-up or contingent work flow tasks in the local work queue. At block 1062, the computing system may send the shared work request disposition and supporting parameters or data to the source pharmacy.

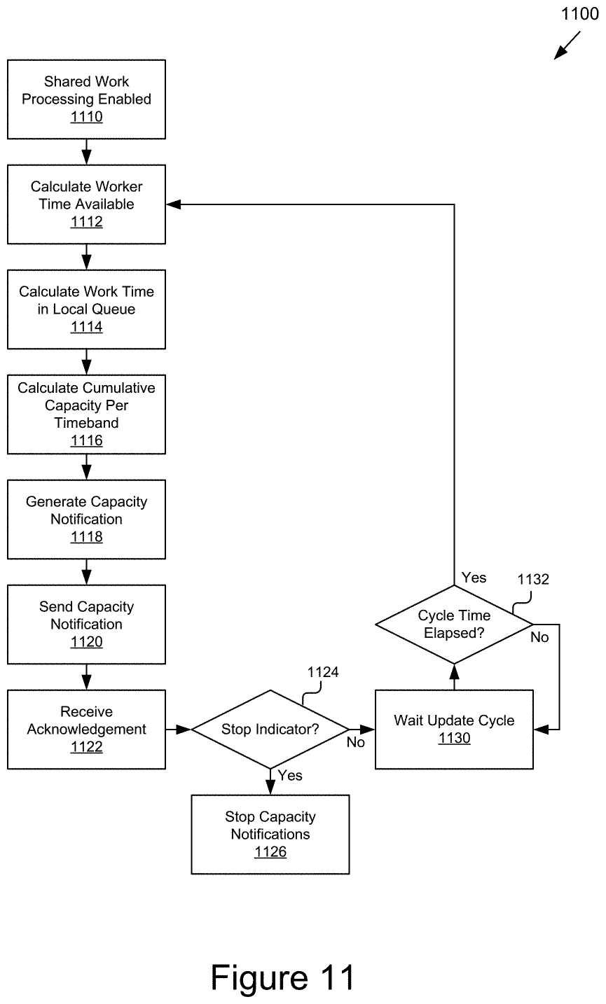

FIG. 11 shows an example method 1100 for updating workload capacity from a processing and/or source location. For example, method 1100 may enable a processing pharmacy computing system, such as computing system 300 in FIG. 3, to send periodic capacity notifications to a work sharing coordinator, such as workload sharing platform 400 in FIGS. 4-6. In some embodiments, capacity notifications may be sent to other computing systems, such as a source pharmacy.

At block 1110, a computing system may have shared work processing enabled. For example, the computing system may include a configuration setting for enabling shared work processing. At block 1112, the computing system may calculate worker time available. For example, the computing system may use pharmacy data related to pharmacy resource scheduling, shifts, and/or presence at the processing location to calculate available worker hours (or other work or productivity metric). At block 1114, the computing system may calculate work time in a local task queue. For example, the computing system may user the tasks and estimated times associated with those tasks to calculate the work time those tasks represent. In some embodiments, the work task time commitments may be projected across time and additional work commitment based on expected work, such as modeled workload based on historical data for the pharmacy location, may be included in committed work capacity values. At block 1116, the computer system may calculate cumulative capacity per timeband using the calculated work time available and capacity commitments in the local work queue. For example, the computing system may include a timebanding model that allocates the work time and associated capacity and commitments into banded periods or time ranges. At block 1118, the computing system may generate a capacity notification based on the preceding calculations.

At block 1120, the computing system may send the capacity notification to one or more recipient systems for capacity modeling. For example, the cumulative capacity and/or underlying data may be formatted in a capacity notification message and sent to the shared work coordinator for use in capacity calculations and assignment scoring. At block 1122, the computing system may receive and acknowledgement that the capacity notification was received. At block 1124, the computing system may check for a stop indicator. For example, the acknowledgement message may include a stop indicator, a separate stop message may be received, or the computing system may initiate a capacity notification stop. If a stop indicator is detected, then method 1100 may proceed to block 1126. If a stop indicator is not detected, then method 1100 may proceed to block 1130. At block 1126, the computing system may stop capacity notifications for a fixed period, until the stop indicator expires, or until a restart indicator is received or generated.

At block 1130, the computing system may wait an update cycle before repeating the notification process. For example, the computing system may include a counter for waiting a predetermined cycle time, such as a period of minutes or seconds provided in a configuration setting for the computing system. At block 1132, the computing system may check whether the cycle time has elapsed. If the cycle time has not elapsed, method 1100 may return to block 1130 and the computing system may continue to wait. If the cycle time has elapsed, method 1100 may return to block 1112 and update the capacity calculations and notification cycle.

As previously discussed, some embodiments may include a user interface at the source pharmacy and/or processing pharmacy based on an existing pharmacy workflow application or pharmacy web portal. Computing systems, such as computing system 200 in FIG. 2 and computing system 300 in FIG. 3 may implement a local work queue interface 1210, such as shown in FIG. 12, and/or a pharmacy work task interface 1310 that may, for example, be displayed on a computer display at the pharmacy.

FIG. 12 illustrates an example graphical user interface 1200 of a local work queue interface 1210 with a shared work dashboard 1220 in accordance with an embodiment. As shown, local work queue interface 1210 may display a list of work tasks 1212, such as production review and data entry review, along with related parameters, such as patient, action time, prescription number, refill number, drug name, quantity, and patient cost. These tasks may be ordered according to priority for selection and completion by a pharmacist. At a source pharmacy, shared tasks may appear in shared work dashboard 1220 as a count against expected return time, grouped in 15-minute intervals starting from the current time. In some embodiments, the summary view of shared work dashboard 1220 may enable navigation to a list view of shared work similar to list of work tasks 1212.

FIG. 13 illustrates an example graphical user interface 1300 of a pharmacy work task interface 1310 displaying a prescription data verification task. As shown, pharmacy work task interface 1310 may display a data entry verification window 1312 to enable a user to navigate, review, and select a disposition for prescription verification. For example, disposition selection options 1320 may allow a pharmacist to select and document a disposition for the work task based on reviewing the displayed prescription data.

In the above description, for purposes of explanation, numerous specific details are set forth in order to provide a thorough understanding of the present disclosure. However, it should be understood that the technology described herein can be practiced without these specific details. Further, various systems, devices, and structures are shown in block diagram form in order to avoid obscuring the description. For instance, various implementations are described as having particular hardware, software, and user interfaces. However, the present disclosure applies to any type of computing device that can receive data and commands, and to any peripheral devices providing services.

In some instances, various implementations may be presented herein in terms of algorithms and symbolic representations of operations on data bits within a computer memory. An algorithm is here, and generally, conceived to be a self-consistent set of operations leading to a desired result. The operations are those requiring physical manipulations of physical quantities. Usually, though not necessarily, these quantities take the form of electrical or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

To ease description, some elements of the system and/or the methods are referred to using the labels first, second, third, etc. These labels are intended to help to distinguish the elements but do not necessarily imply any particular order or ranking unless indicated otherwise.

It should be borne in mind, however, that all of these and similar terms are to be associated with the appropriate physical quantities and are merely convenient labels applied to these quantities. Unless specifically stated otherwise as apparent from the following discussion, it is appreciated that throughout this disclosure, discussions utilizing terms including “processing,” “computing,” “calculating,” “determining,” “displaying,” or the like, refer to the action and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

Various implementations described herein may relate to an apparatus for performing the operations herein. This apparatus may be specially constructed for the required purposes, or it may comprise a general-purpose computer selectively activated or reconfigured by a computer program stored in the computer. Such a computer program may be stored in a computer readable storage medium, including, but is not limited to, any type of disk including floppy disks, optical disks, CD-ROMs, and magnetic disks, read-only memories (ROMs), random access memories (RAMs), EPROMs, EEPROMs, magnetic or optical cards, flash memories including USB keys with non-volatile memory or any type of media suitable for storing electronic instructions, each coupled to a computer system bus.

The technology described herein can take the form of an entirely hardware implementation, an entirely software implementation, or implementations containing both hardware and software elements. For instance, the technology may be implemented in software, which includes but is not limited to firmware, resident software, microcode, etc. Furthermore, the technology can take the form of a computer program object accessible from a computer-usable or computer-readable medium providing program code for use by or in connection with a computer or any instruction execution system. For the purposes of this description, a computer-usable or computer readable medium can be any non-transitory storage apparatus that can contain, store, communicate, propagate, or transport the program for use by or in connection with the instruction execution system, apparatus, or device.

A data processing system suitable for storing and/or executing program code may include at least one processor coupled directly or indirectly to memory elements through a system bus. The memory elements can include local memory employed during actual execution of the program code, bulk storage, and cache memories that provide temporary storage of at least some program code in order to reduce the number of times code must be retrieved from bulk storage during execution. Input or I/O devices (including but not limited to keyboards, displays, pointing devices, etc.) can be coupled to the system either directly or through intervening I/O controllers.

Network adapters may also be coupled to the system to enable the data processing system to become coupled to other data processing systems, storage devices, remote printers, etc., through intervening private and/or public networks. Wireless (e.g., Wi-Fi™) transceivers, Ethernet adapters, and Modems, are just a few examples of network adapters. The private and public networks may have any number of configurations and/or topologies. Data may be transmitted between these devices via the networks using a variety of different communication protocols including, for example, various Internet layer, transport layer, or application layer protocols. For example, data may be transmitted via the networks using transmission control protocol/Internet protocol (TCP/IP), user datagram protocol (UDP), transmission control protocol (TCP), hypertext transfer protocol (HTTP), secure hypertext transfer protocol (HTTPS), dynamic adaptive streaming over HTTP (DASH), real-time streaming protocol (RTSP), real-time transport protocol (RTP) and the real-time transport control protocol (RTCP), voice over Internet protocol (VOIP), file transfer protocol (FTP), WebSocket (WS), wireless access protocol (WAP), various messaging protocols (SMS, MMS, XMS, IMAP, SMTP, POP, WebDAV, etc.), or other known protocols.

Finally, the structure, algorithms, and/or interfaces presented herein are not inherently related to any particular computer or other apparatus. Various general-purpose systems may be used with programs in accordance with the teachings herein, or it may prove convenient to construct more specialized apparatus to perform the required method blocks. The required structure for a variety of these systems will appear from the description above. In addition, the specification is not described with reference to any particular programming language. It will be appreciated that a variety of programming languages may be used to implement the teachings of the specification as described herein.

The foregoing description has been presented for the purposes of illustration and description. It is not intended to be exhaustive or to limit the specification to the precise form disclosed. Many modifications and variations are possible in light of the above teaching. As will be understood by those familiar with the art, the specification may be embodied in other specific forms without departing from the spirit or essential characteristics thereof. Likewise, the particular naming and division of the modules, routines, features, attributes, methodologies and other aspects are not mandatory or significant, and the mechanisms that implement the specification or its features may have different names, divisions and/or formats.

Furthermore, the modules, routines, features, attributes, methodologies and other aspects of the disclosure can be implemented as software, hardware, firmware, or any combination of the foregoing. Also, wherever a component, an example of which is a module, of the specification is implemented as software, the component can be implemented as a standalone program, as part of a larger program, as a plurality of separate programs, as a statically or dynamically linked library, as a kernel loadable module, as a device driver, and/or in every and any other way known now or in the future. Additionally, the disclosure is in no way limited to implementation in any specific programming language, or for any specific operating system or environment.

What is claimed is:

1. A system comprising:
an enterprise computing device configured to generate and assign a shared work request for a prescription verification task, wherein the prescription verification task is a prerequisite task for a production task at a source pharmacy;
a processing pharmacy computing device at a processing pharmacy configured to generate a processing graphical user interface displaying the prescription verification task assigned by the enterprise computing system, wherein the processing graphical user interface is configured to display prescription verification data and a plurality of selectable disposition options for the prescription verification task; and
a source pharmacy computing device at the source pharmacy configured to generate a production graphical user interface displaying the production task in response to receiving a disposition of the prescription verification task.

2. The system of claim 1, wherein the processing graphical user interface is further configured to:
display, in a work task interface, local work tasks corresponding to production tasks at the processing pharmacy; and
responsive to receiving the assigned shared work request, display a shared work task in the work task interface for the prescription verification task.

3. The system of claim 1, wherein the processing graphical user interface is further configured to display:
the prescription verification data in a first portion of the processing graphical user interface; and
the plurality of selectable disposition options for the prescription verification in a second portion of the processing graphical user interface.

4. The system of claim 1, wherein the production graphical user interface is configured to display a shared work dashboard configured to display shared work indicators for at least one shared work request.

5. The system of claim 4, wherein:
the shared work dashboard is further configured to display shared work indicators for a plurality of shared work requests; and
the shared work indicators are displayed as at least one count value against expected return time.

6. The system of claim 5, wherein the at least one count value includes a plurality of count values arranged in predetermined time intervals for groups of shared work requests with return times in that predetermined time interval.

7. The system of claim 6, wherein:
the enterprise computing device is further configured to:
calculate a work delivery time for the shared work request; and
calculate a recall deadline for the prescription verification task assigned to the processing pharmacy;
the work delivery time includes a first work completion offset to enable additional work tasks to be completed at the source pharmacy in response to disposition of the prescription verification task;
the recall deadline includes a second work completion offset to enable the prescription verification task to be completed at the source pharmacy in response to terminating the shared work request; and
the return times are based on the recall deadline for corresponding shared work requests.

8. The system of claim 1, wherein:
the source pharmacy computing device is further configured to:
receive a prescription for the production task;
process the prescription to generate work parameters for the prescription verification task; and
send, to the enterprise computing device, a work request including the work parameters;
the enterprise computing device is further configured to generate the shared work request based on the work request including the work parameters; and
the prescription verification data displayed on the processing graphical user interface is based on the work parameters.

9. The system of claim 8, wherein:
the source pharmacy computing device is further configured to:
identify a protected data resource at the source pharmacy; and
send the protected data resource to the processing pharmacy computing device;
the processing pharmacy computing device is further configured to receive the protected data resource from the source pharmacy computing device; and
the prescription verification data displayed on the processing graphical user interface includes the protected data resource for the prescription verification task.

10. The system of claim 1, wherein:
the enterprise computing device is further configured to:
receive a work request from the source pharmacy;
process a set of qualification rules in response to receiving the work request; and
identify the processing pharmacy using a set of assignment rules in response to a successful qualification of the work request; and
generating and assigning the shared work request for the prescription verification task to the processing pharmacy is based on the work request from the source pharmacy.

11. A computer-implemented method comprising:
receiving, by a processing pharmacy computing device at a processing pharmacy, a shared work request for a prescription verification task, wherein the prescription verification task is a prerequisite task for a production task at a source pharmacy that is different than the processing pharmacy;
generating, at the processing pharmacy computing device, a processing graphical user interface displaying the prescription verification task from the shared work request, wherein the processing graphical user interface is configured to display prescription verification data and a plurality of selectable disposition options for the prescription verification task; and
sending, to a source pharmacy computing device at the source pharmacy, a disposition of the prescription verification task, wherein the source pharmacy computing device is configured to generate a production graphical user interface displaying the production task in response to receiving the disposition of the prescription verification task.

12. The computer-implemented method of claim 11, further comprising:
displaying, in a work task interface of the processing graphical user interface, local work tasks corresponding to production tasks at the processing pharmacy; and
responsive to receiving the shared work request, displaying a shared work task in the work task interface for the prescription verification task.

13. The computer-implemented method of claim 11, further comprising:
displaying the prescription verification data in a first portion of the processing graphical user interface; and
displaying the plurality of selectable disposition options for the prescription verification in a second portion of the processing graphical user interface.

14. The computer-implemented method of claim 11, further comprising:
displaying, in the production graphical user interface, a shared work dashboard configured to display shared work indicators for at least one shared work request.

15. The computer-implemented method of claim 14, further comprising:
displaying, in the shared work dashboard, shared work indicators for a plurality of shared work requests, wherein the shared work indicators are displayed as at least one count value against expected return time.

16. The computer-implemented method of claim 15, wherein the at least one count value includes a plurality of count values arranged in predetermined time intervals for groups of shared work requests with return times in that predetermined time interval.

17. The computer-implemented method of claim 16, further comprising:
calculating a work delivery time for the shared work request; and
calculating a recall deadline for the prescription verification task assigned to the processing pharmacy, wherein:
the work delivery time includes a first work completion offset to enable additional work tasks to be completed at the source pharmacy in response to disposition of the prescription verification task;
the recall deadline includes a second work completion offset to enable the prescription verification task to be completed at the source pharmacy in response to terminating the shared work request; and
the return times are based on the recall deadline for corresponding shared work requests.

18. The computer-implemented method of claim 11, further comprising:
receiving, by the source pharmacy computing device, a prescription for the production task;
processing, by the source pharmacy computing device, the prescription to generate work parameters for the prescription verification task; and
sending, by the source pharmacy computing device, the shared work request including the work parameters, wherein the prescription verification data displayed on the processing graphical user interface is based on the work parameters.

19. The system of claim 18, further comprising:
receiving, by an enterprise computing device, the shared work request from the source pharmacy;
processing, by the enterprise computing device, a set of qualification rules in response to receiving the shared work request;
identifying, by the enterprise computing device, the processing pharmacy using a set of assignment rules in response to a successful qualification of the shared work request; and
assigning, by the enterprise computing device, the shared work request for the prescription verification task to the processing pharmacy.

20. A system comprising:
a processor;
a memory; and
a display configured for displaying a production graphical user interface for a source pharmacy, wherein the production graphical user interface is configured to:
display, in a work task interface of the production graphical user interface, local work tasks corresponding to production tasks at the source pharmacy; and
overlay, on the work task interface, a shared work dashboard configured to display shared work indicators for at least one shared work request from the source pharmacy.
